(19) United States
(12) Patent Application Publication
(10) Pub. No.: US 2007/0074295 A1
(43) Pub. Date: Mar. 29, 2007

(54) Schizophrenia related gene

(75) Inventors: Susan Cochran, Iruma Saitama (JP); Gary Paterson, Glasgow (GB); Yoshitaka Ohashi, Iruma Saitama (JP); Brian Morris, Glasgow (GB); Judith Pratt, Glasgow (GB)

Correspondence Address: FISH & NEAVE IP GROUP;ROPES & GRAY LLP, ONE INTERNATIONAL PLACE, BOSTON, MA, 02110-2624, US

(73) Assignee: Mitsubishi Pharma Corporation, Osaku (JP)

(21) Appl. No.: 11/518,868

(22) Filed: Sep. 11, 2006

Related U.S. Application Data
(63) Continuation of application No. 10/240,154, filed on Apr. 29, 2003, now abandoned.

(30) Foreign Application Priority Data
Mar. 31, 2000 (GB) 0007880.8
May 26, 2000 (GB) 0012768.8

(51) Int. Cl. A01K 67/027; C12Q 1/68; G01N 33/53; C07H 21/04; C12P 21/06; A61K 38/17; A61K 48/00; C07K 14/705

(52) U.S. Cl. 800/3; 435/6; 536/23.2; 435/69.1; 435/320.1; 435/325; 435/7.1; 530/350; 530/388.22; 514/44; 800/14; 800/18

(57) ABSTRACT

There are provided isolated polynucleotide fragments for use in diagnosing and/or developing treatments for schizophrenia. Further provided is a method for diagnosing schizophrenia using one or more polynucleotides disclosed herein. Also provided is a method for screening a compound which regulates expression of a schizophrenia-related gene. Also provided is a chronic animal model of schizophrenia that mimics the functional deficits observed in patients and methods for producing the animal model comprising the administration of PCP to the animal.

### CROSS REFERENCE TO RELATED APPLICATIONS

This application is a continuation of U.S. application Ser. No. 10/240,154, filed Apr. 29, 2003, which is a national stage filing under 35 U.S.C. 371 of PCT application PCT/GB01/01486, filed Apr. 2, 2001, which claims priority from United Kingdom Application Nos. 0007880.8, filed Mar. 31, 2000 and 0012768.8, filed May 26, 2000, the specifications of all of which are incorporated by reference herein. PCT Application PCT/GB01/01486 was published under PCT Article 21(2) in English.

The present invention relates to the identification of genes postulated to be involved and/or associated with schizophrenia. The present invention also relates to the development of a chronic animal model which mimics functional deficits in schizophrenia and to the use of the model in drug screening and identification of genes/proteins associated with schizophrenia, as well as particular identified genes and their use in therapy/diagnosis of schizophrenia.

Schizophrenia is a devastating mental illness which affects 1% of the world population, the aetiology of which remains elusive. To date, there is a poor understanding of the genes involved and no chronic animal models of schizophrenia have been developed which imitate all the characteristics of the disease.

One of the goals of modern antipsychotic drug development is to produce a drug which is more effective in ameliorating the negative symptoms and cognitive deficits characteristic of schizophrenia than existing therapies. Although typical and atypical antipsychotic drugs, such as haloperidol and clozapine, are effective in attenuating the positive symptoms, they are ineffective (haloperidol) or minimally effective (clozapine) against the negative symptoms and cognitive dysfunction associated with the disease (Goldberg, T. et al). The development of improved antipsychotic drugs which will have superior action against the negative symptoms and cognitive dysfunction has been severely hampered by the lack of knowledge of which genes are involved and/or associated with schizophrenia, or lack of an animal model which accurately models these symptoms.

Many putative models of schizophrenia have been described to date. These range from developmental models (Lillrank et al), social isolation (Jones, G. H. et al) or social interaction (Sams-Dodd, F. et al) models to pharmacological models (Snyder, S. H. et al). The major drawbacks of the present pharmacological models of schizophrenia are that they are based on acute administration of the drugs. The models involve administering the drug to produce the psychotic state, but in order to test the activity of antipsychotic drugs, they are administered before the animal is exposed to amphetamine or Phencyclidine (PCP). This would be tantamount to administering an antipsychotic drug to a patient before the onset of schizophrenia. The models also do not account for the fact that antipsychotic treatment can take up to a month to have beneficial effects against the disease. Thus, the current models of schizophrenia fail to accurately mimic the clinical profile of the disease.

Moreover, little is known about the genes, or more specifically any alteration of expression/mutation of genes in a patient suffering from schizophrenia.

It is therefore amongst the objects of the present invention to obviate and/or mitigate at least one of the aforementioned disadvantages.

The present invention is based in part on the development of a chronic animal model of schizophrenia using the drug phencyclidine (PCP) and the use of this model to identify genes thought to be involved and/or associated with schizophrenia. Although PCP has been known for many years to produce schizophrenic-like symptoms in man and also to worsen the psychotic state in schizophrenics (Allen, R. M. et al), it has hitherto not been used to develop a chronic animal model of schizophrenia that mimics the functional deficits observed in patients.

The present invention is also based in part on the elucidation of genes which are differentially expressed in the blood of schizophrenic patients.

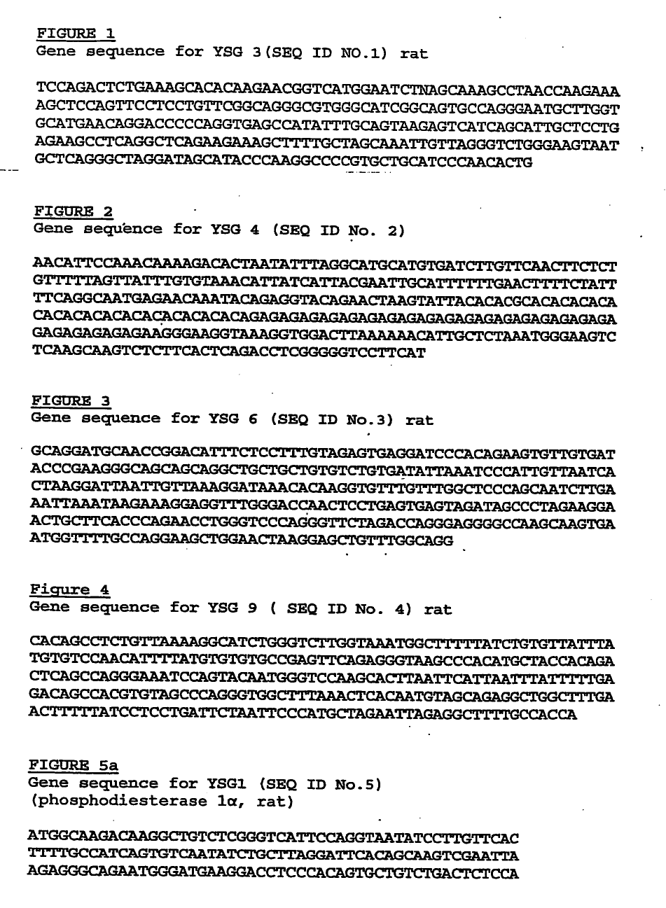

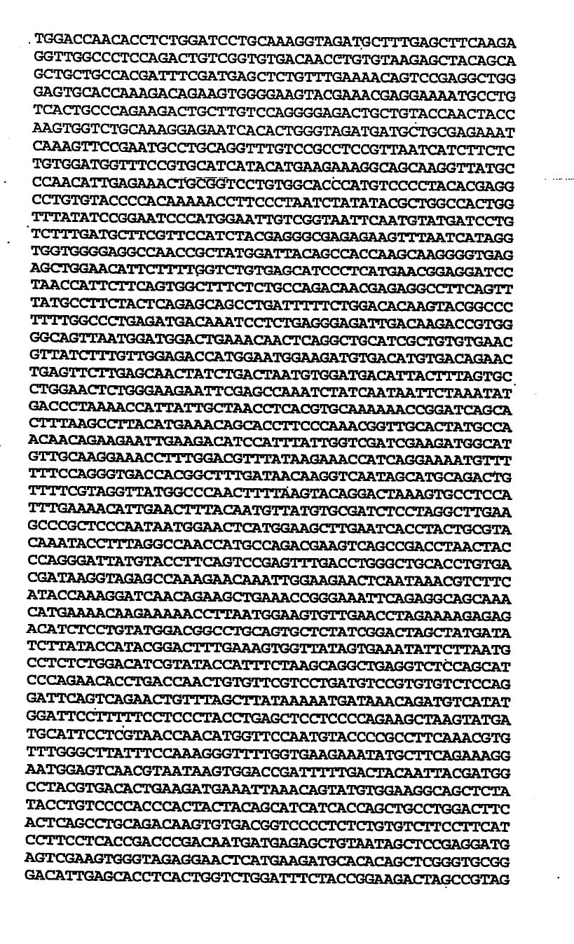

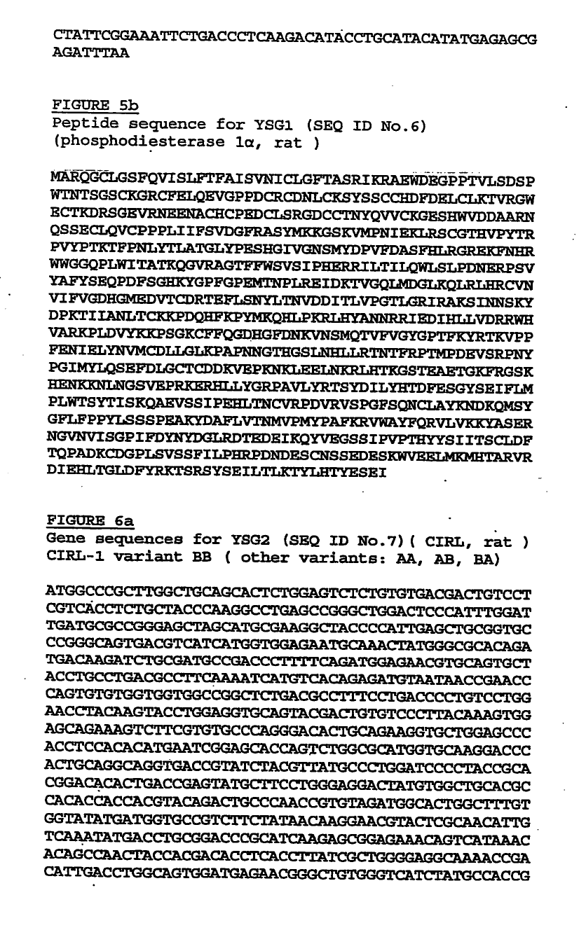

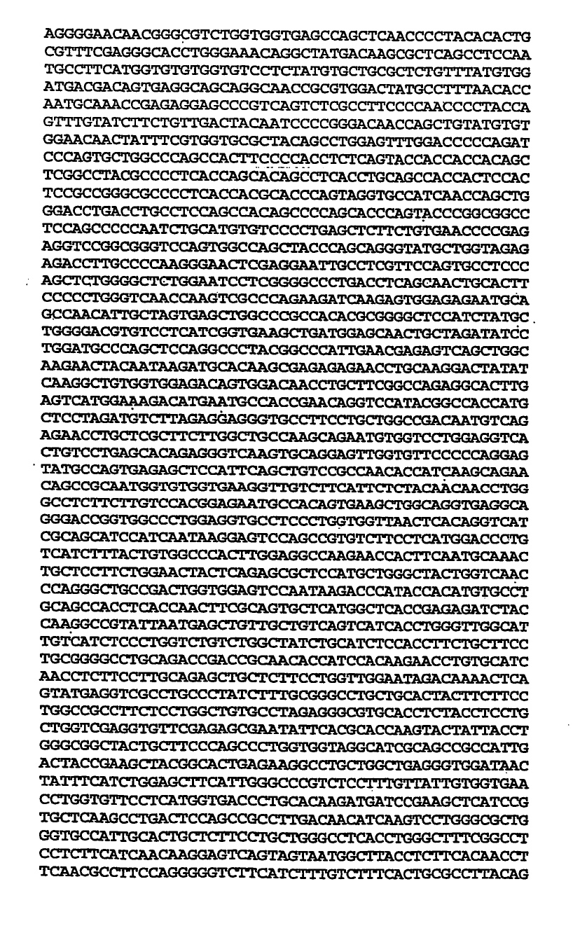

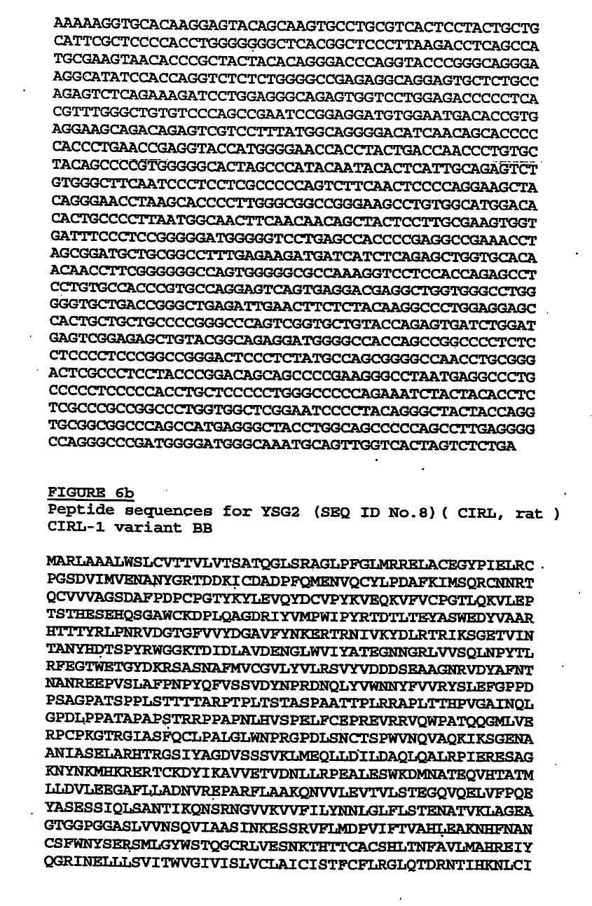

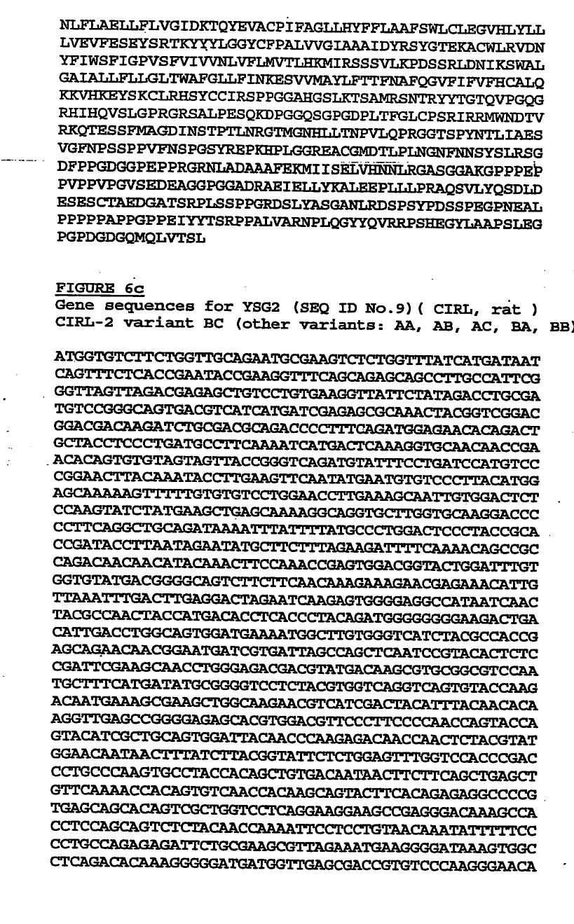

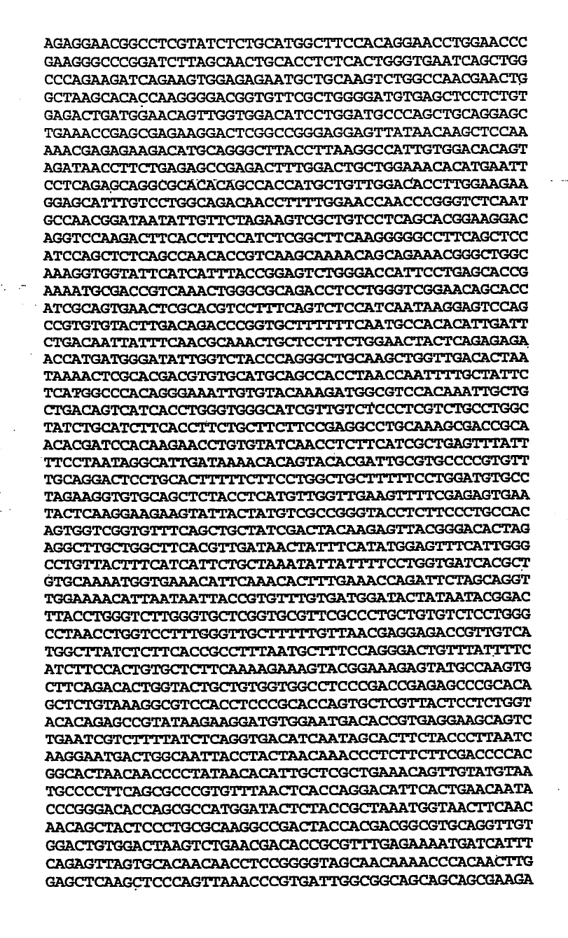

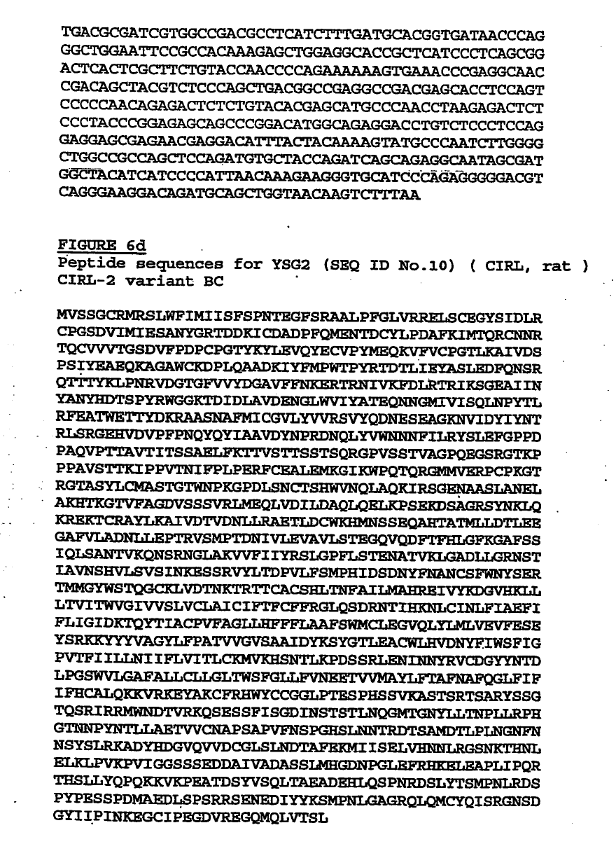

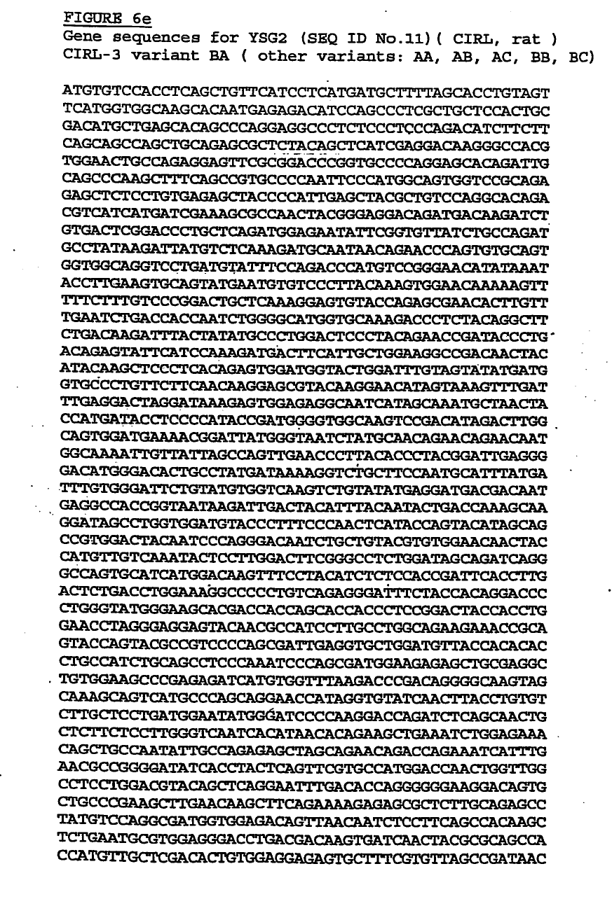

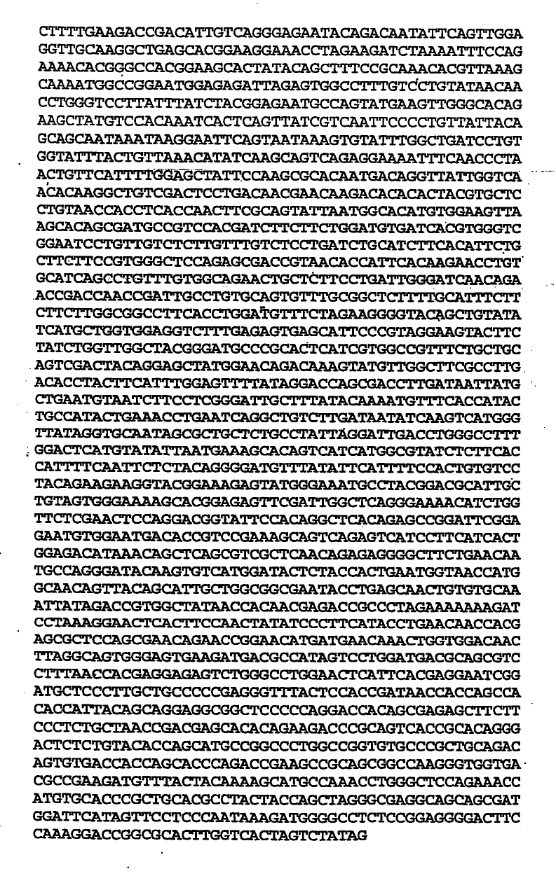

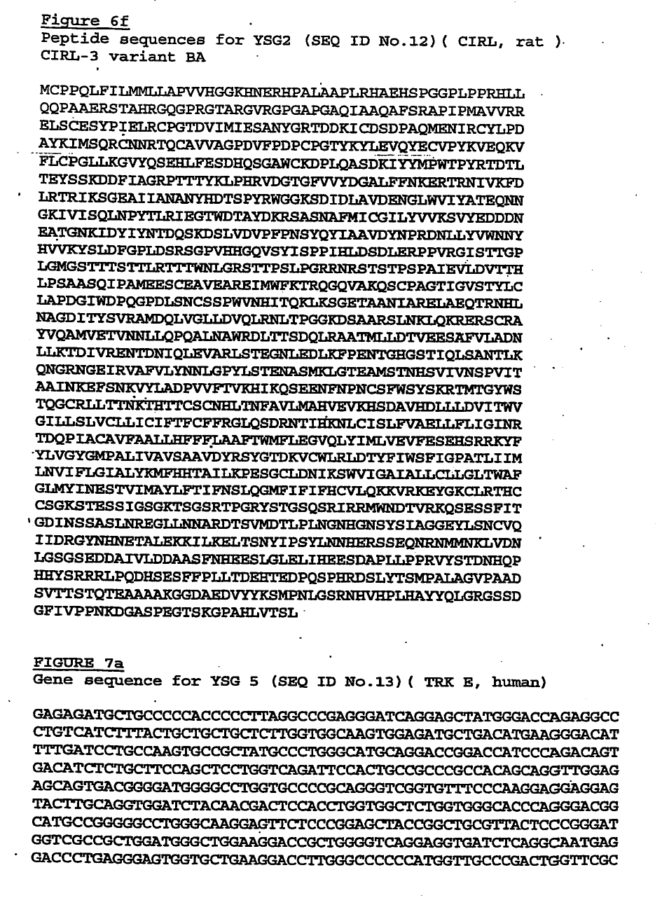

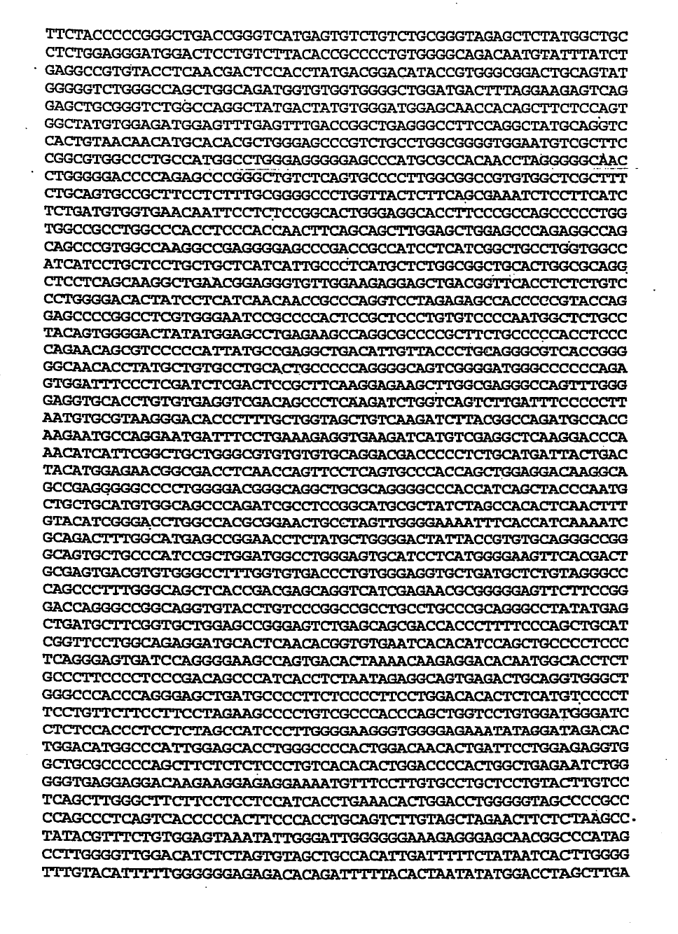

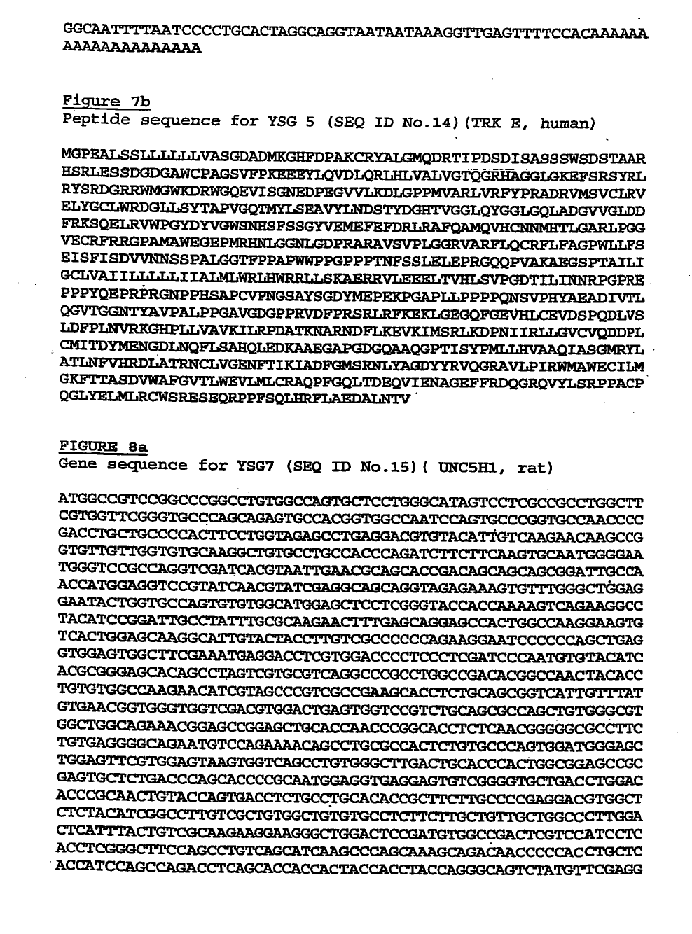

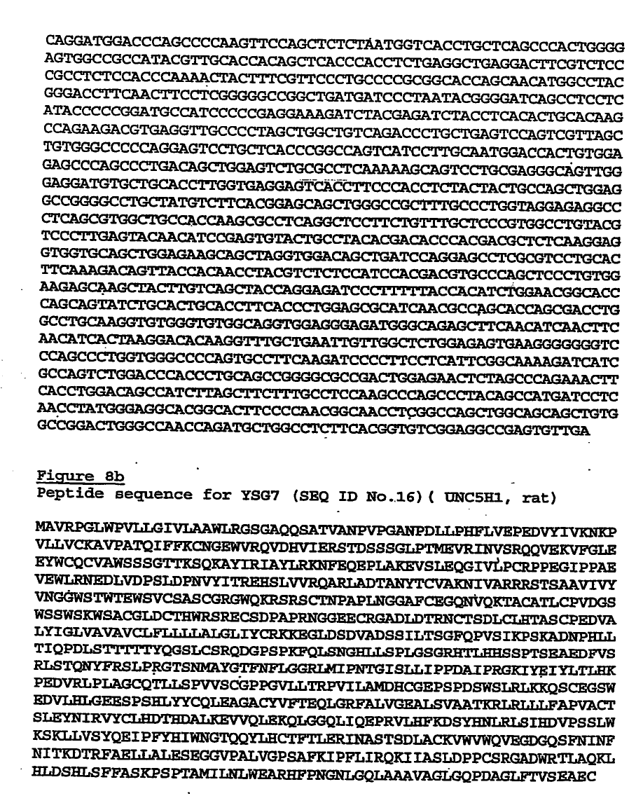

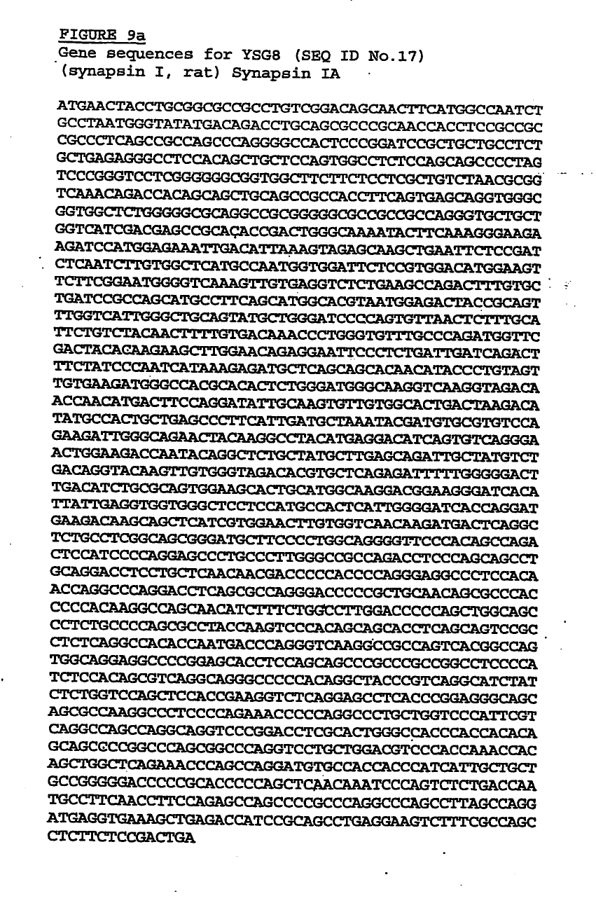

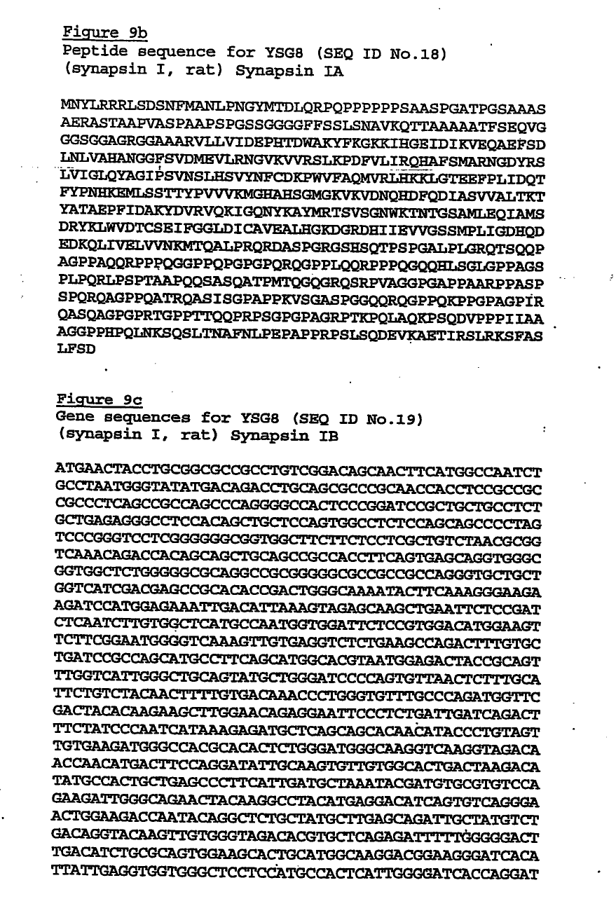

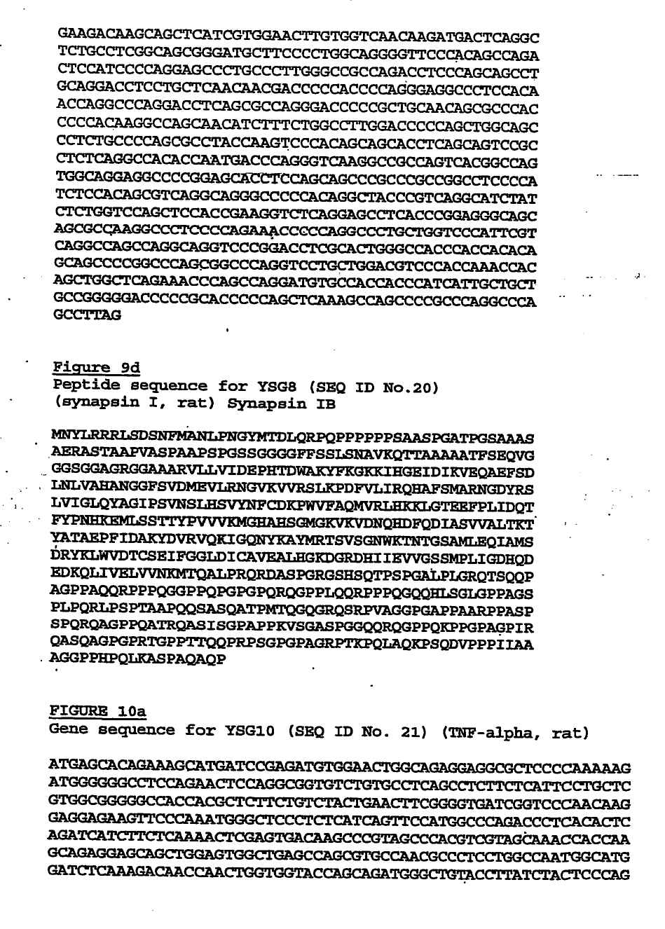

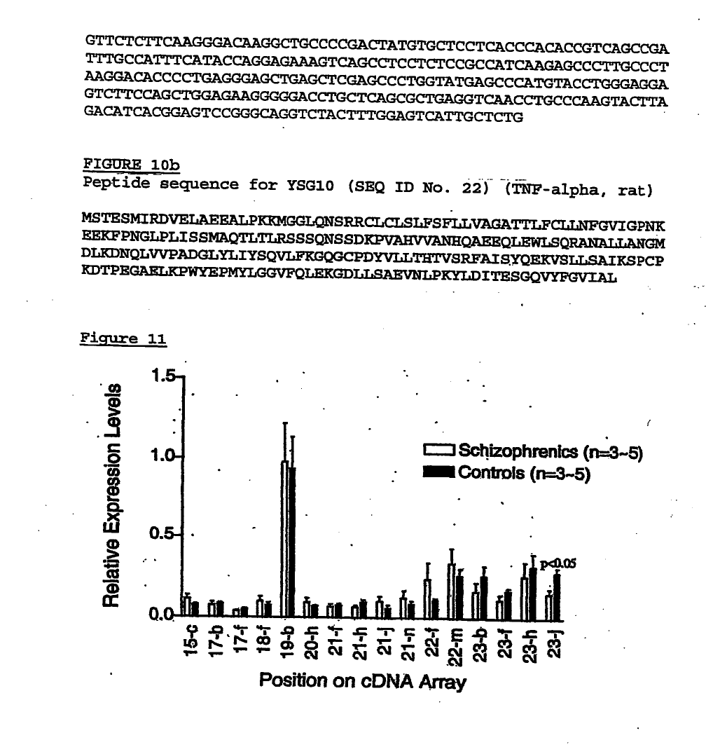

Thus, according to a first aspect, there are provided isolated polynucleotide fragments for use in diagnosing and/or developing treatments for schizophrenia. The isolated polynucleotide fragments are shown in the attached FIGS. 1, 2, 3, 4, 5a, 6a, 6c, 6e, 7a, 8a, 9a, 9c and 10a. The inventors have presently identified 10 genes which have been observed to be differentially expressed in the animal model disclosed herein or in blood samples from schizophrenic patients. The genes have been designated YSG1-10. The YSG3 (FIG. 1: SEQ ID No. 1), YSG4 (FIG. 2: SEQ ID No. 2), YSG6 (FIG. 3: SEQ ID No. 3) and YSG9 (FIG. 4: SEQ ID No. 4) are shown to be novel sequences based on database screening. The remaining sequences are known genes not however previously being associated with schizophrenia; YSG1 (FIG. 5a: SEQ ID No. 5) relates to phosphodiesterase 1α YSG2 (FIGS. 6a, 6c, 6e: SEQ ID Nos. 7, 9 & 11, respectively) relates to calcium-independent alpha-latrotoxin receptor (CIRL 1, 2 & 3); YSG5 (FIG. 7a: SEQ ID No. 13) relates to epithelial discoidin domain receptor 1, trkE; YSG7 (FIG. 8a: SEQ ID No. 15) relates to netrin receptor UNC5H1; YSG8 (FIGS. 9a, 9c: SEQ ID Nos. 17 & 19, respectively) relates to synapsins 1A and 1B; and YSG10 (Figure 10a: SED ID No. 21) relates to TNFα.

Thus the present invention provides a polynucleotide having DNA sequence represented by SEQ ID No, 1; a polynucleotide having DNA sequence represented by SEQ ID No. 2; a polynucleotide having DNA sequence represented by SEQ ID No. 3; or a polynucleotide having DNA sequence represented by SEQ ID No. 4.

The present invention also provides a method for diagnosing schizophrenia which comprises using one or more polynucleotides selected from the group consisting of SEQ ID No, 1, SEQ ID No. 2, SEQ ID No. 3, SEQ ID No, 4, SEQ ID No, 5 (PDE1α), SEQ ID No. 7, 9 & 11 (CIRL 1, 2 & 3), SEQ ID NO, 13 (trkE), SEQ ID No, 15 (netrin receptor), SEQ ID. No. 17 & 19 (synapsin 1A/1B) and SEQ ID No. 21 (TNFα) as indicator(s)

The above described polynucleotide fragments have been discovered to be differentially expressed in a chronic animal model as described herein or in the blood of schizophrenic patients and are postulated therefore to be putatively involved and/or associated with schizophrenia.

“Polynucleotide fragment” as used herein refers to a polymeric form of nucleotides of any length, both to ribonucleic acid sequences and to deoxyribonucleic acid sequences. In principle, this term refers to the primary structure of the molecule. Thus, the term includes double stranded and single stranded DNA, as well as double and single stranded RNA, and modifications thereof.

In a further aspect the present invention provides polynucleotide fragments encoding polypeptides for use in diagnosing and/or developing treatments for schizophrenia.

In particular the polypeptides are shown in FIGS. 5b, 6b, 6d, 6f, 7b, 8b, 9b, 9d and 10b, relating to SEQ ID Nos. 6, 8, 10, 12, 14, 16, 18, 20 & 22.

In general, the term “polypeptide” refers to a molecular chain of amino acids with a biological activity. It does not refer to a specific length of the product, and if required can be modified in vivo and/or in vitro, for example by glycosylation, myristoylation, amidation, carboxylation or phosphorylation; thus inter alia peptides, oligopeptides and proteins are included. The polypeptides disclosed herein may be obtained by synthetic or recombinant techniques known in the art.

Thus, the term extends to cover for example polypeptides obtainable from various transcripts and splice variants of these transcripts from a particular gene.

It will be understood that for the polynucleotide fragments and polypeptide sequences presented herein, natural variations can exist between individuals. These variations may be demonstrated by nucleotide and/or amino acid differences in the overall sequence or by deletions, substitutions, insertions or inversions of nucleotides or amino acids in said sequences.

As is well known in the art, the degeneracy of the genetic code permits substitution of bases in a codon resulting in another codon for the same amino acid, Consequently, it is clear that any such derivative nucleotide sequence based on the sequences disclosed herein are also included in the scope of the present invention.

Thus, the present invention further includes nucleotide and/or polypeptide sequences having at least 80%, particularly at least 90%, and especially at least 95% homology or similarity with the sequences shown in the attached Figures.

The present invention also includes nucleotide sequences similar to the polynucleotide sequences disclosed herein. It is understood that similar sequences include sequences which remain hybridised to the polynucleotide sequences of the present invention under stringent conditions. Typically a test similar sequence and a polynucleotide sequence of the present invention are allowed to hybridise for a specified period of time generally at a temperature of between 50 and 70^{N}C in double strength SSC (2×NaCl 17.5 g/l and sodium citrate (SC) at 8.8 g/l) buffered saline containing 0.1% sodium dodecyl sulphate (SDS) followed by rinsing of the support at the same temperature but with a buffer having a reduced SSC concentration. Depending upon the degree of similarity of the sequences, such reduced concentration buffers are typically single strength SSC containing 0.1% SDS, half strength SSC containing 0.1% SDS and one tenth strength SSC containing 0.1% SDS. Sequences having the greatest degree of similarity are those the hybridisation of which is least affected by washing in buffers of reduced concentration. It is most preferred that the similar and inventive sequences are so familiar that the hybridisation between them is substantially unaffected by washing or incubation in one tenth strength SSC containing 0.1% SDS.

Furthermore, fragments derived from the polynucleotide fragments depicted in the Figures may be used.

Moreover, fragments derived from the encoded polypeptides are also encompassed by the present invention.

All such modifications mentioned above resulting in such derivatives of the polypeptides are covered by the present invention so long as the characteristic polypeptide properties remain substantially unaffected in essence.

The information presented herein can be used to genetically manipulate the sequences or derivatives thereof, for example to clone the sequences by recombinant DNA techniques generally known in the art. Cloning of homologous sequences from other species of mammal, and in particular humans, may be performed with the information disclosed herein by widely known techniques; for example, oligonucleotides may be designed to a consensus region and/or functional domains of the sequences shown in the Figures and such oligonucleotides, and/or the polymerase chain reaction products generated using these oligonucleotide primers, can be used as probes for cloning homologous sequences from other organisms, for example by polymerase chain reaction or by hybridisation.

The polynucleotide fragments of the present invention may be linked to expression control sequences. Such control sequences may comprise promoters, operators, inducers, ribosome binding sites etc. Suitable control sequences for a given host may be selected by those of ordinary skill in the art.

A nucleotide sequence according to the present invention can be ligated to various expression-controlling DNA sequences, resulting in a so-called recombinant nucleic acid molecule. Thus the present invention also includes an expression vector comprising an expressible nucleotide sequence. Said recombinant nucleic acid molecule can then be used for transformation of a suitable host.

Such recombinant nucleic acid molecules are preferably derived from for example, plasmids, or from nucleic acid sequences present in bacteriophages or viruses and are termed vector molecules.

Specific vectors which can be used to clone nucleotide sequences according to the invention are known in the art (eg. Rodriguez RL and DT Denhardt, editors, Vectors: A survey of molecular cloning vectors and their uses, Butterworths, 1988).

The methods to be used for the construction of a recombinant nucleic acid molecule according to the invention are known to those of ordinary skill in the art and are inter alia set forth in Sambrook et al, Molecular Cloning: A Laboratory Manual, Cold Spring Harbour Laboratory, 1989.

The present invention also relates to a transformed cell comprising the polynucleotide fragments of the present invention, in expressible form, if appropriate. “Transformation”, as used herein, refers to the introduction of a heterologous nucleic acid sequence into a host cell in vivo, ex vivo or in vitro irrespective of the method used, for example, by calcium phosphate coprecipitation, direct uptake, electroporation or transduction.

The heterologous nucleic acid sequence may be maintained through autonomous replication or alternatively may be integrated into the host's genome. The recombinant DNA molecules preferably are provided with appropriate control sequences, compatible with the designated host which can regulate the expression of the inserted nucleic acid sequence.

The most widely used hosts for expression of recombinant nucleic acid molecules are bacteria, yeast, insect cells and mammalian cells. Each system has advantages and disadvantages in terms of the vector used, potential ease of production and purification of a recombinant polypeptide and authenticity of product in terms of tertiary structure, glycosylation state, biological activity and stability and will be a matter of choice for the skilled addressee.

In addition to expressing the polynucleotide fragments of the present invention, in certain circumstances, it is advantageous to substantially prevent or reduce the expression or activity of the polynucleotide fragments in a cell or host. Thus, according to a further aspect of the invention, there is provided an antisense nucleotide fragment complementary to a polynucleotide fragment or subfragment of the present invention. Included in the scope of “antisense nucleotide fragment” is the use of synthetic oligonucleotide sequences, or of equivalent chemical entities known to those skilled in the art, for example, peptide nucleic acids. Also provided is a nucleotide fragment comprising a nucleotide sequence which, when transcribed by the cell, produces such an antisense fragment. Typically antisense RNA fragments will be provided which bind to complementary mRNA fragments to form RNA double helices, allowing RNAse H to cleave the molecule and rendering it incapable of being translated by the cell into polypeptides.

A further aspect of the present invention provides antibodies specific to the polypeptides of the present invention or epitopes thereof. Production and purification of antibodies specific to an antigen is a matter of ordinary skill, and the methods to be used are clear to those skilled in the art. The term antibodies can include, but is not limited to polyclonal antibodies, monoclonal antibodies (mAbs), humanised or chimeric antibodies, single chain antibodies, Fab fragments, F(ab′)₂ fragments, fragments produced by a Fab expression library, antiidiotypic (anti-Id) antibodies, and epitope binding fragments of any of the above. Such antibodies may be used in modulating the expression or activity of the particular polypeptide, or in detecting said polypeptide in vivo or in vitro.

The present invention further provides a recombinant or synthetic polypeptide for the manufacture of reagents for use as therapeutic agents in the treatment of schizophrenia. In particular, the invention provides pharmaceutical compositions comprising the recombinant or synthetic polypeptide together with a pharmaceutically acceptable carrier therefor.

The present invention also relates to methods for prognostic and/or diagnostic evaluation of schizophrenia and/or for the identification of subjects who are predisposed to schizophrenia, for example by examination of allelic variation by determination of the expression or sequence of the genes identified herein in an individual. Furthermore, the invention provides methods for evaluating the efficacy of drugs for such disorders, and monitoring the progress of patients involved in clinical trials for the treatment of such disorders.

Thus the invention further provides methods for the identification of compounds which modulate the expression of the polynucleotide fragments and/or the activity of polypeptide sequences identified herein. Such identified compounds may be used in the treatment of schizophrenia.

Thus there is provided a method for screening a compound which regulates expression of a schizophrenia-related gene(s), which comprises:

(a) bringing a test compound into contact with transformed cell which enables expression of a gene selected from the group consisting of SEQ ID No. 1, SEQ ID No. 2, SEQ ID No. 3, SEQ ID No. 4, SEQ ID No. 5 (PDE1α), SEQ ID No. 7, 9 & 11 (CIRL 1, 2 & 3), SEQ ID No. 13 (trkE), SEQ ID No. 15 (netrin receptor), SEQ ID No. 17 & 19 (Synapsin 1A/1B) and SEQ ID No. 21 (TNFα),

(b) detecting an expression of schizophrenia-relating factor in said cell, and

(c) selecting a compound which promotes or suppresses an induction of the schizophrenia-relating factor in comparison with a control (vehicle).

There is also provided a method for measuring an anti-schizophrenic effects of a compound using the animal model of the present invention, which comprises:

(a) measuring local cerebral glucose utilisation (LCGU), or detecting an expression level of one or more genes represented by the group consisting of SEQ ID No. 1, SEQ ID No, 2, SEQ ID No. 3, SEQ ID No. 4, SEQ ID No. 5 (PDE 1α), SEQ ID No. 7, 9 & 11 (CIRL 1, 2 & 3), SEQ ID No. 13 (trkE), SEQ ID No. 15 (netrin receptor), SEQ ID No. 17 & 19 (synapsin 1A/1B) and SEQ ID No. 21 (TNFα), and

(b) comparing with a control group.

The biological function of the genes identified herein can be more directly assessed by utilizing relevant in vivo and in vitro systems. In vivo systems can include, but are not limited to, animal systems which naturally exhibit the symptoms of schizophrenia, or ones which have been engineered to exhibit such symptoms, as for example the model described herein. Further, such systems can include, but are not limited to transgenic animal systems. In vivo systems can include, but are not limited to, cell-based systems comprising the identified gene/polypeptide expressing cell types. The cells can be wild type cells, or can be non-wild type cells containing modifications known or suspected of contributing to schizophrenia.

In further characterising the biological function of said identified gene(s), the expression of said identified gene(s) can be modulated within the in vivo and/or in vitro systems, i.e. either overexpressed or underexpressed in, for example, transgenic animals and/or cell lines, and its subsequent effect on the system can then be assayed.

Alternatively, the activity of the product of the identified gene can be modulated by either increasing or decreasing the level of activity in the in vivo and/or in vitro system of interest, and its subsequent effect then assayed.

The information obtained through such characterisations can suggest relevant methods for the treatment or control of schizophrenia. For example, relevant treatment can include a modulation of gene expression and/or gene product activity. Characterisation procedures such as those described herein can indicate whether such modulation should be positive or negative. As used herein, “positive modulation” refers to an increase in gene expression or activity of the gene or gene product of interest. “Negative modulation”, as used herein, refers to a decrease in gene expression or activity.

In vitro systems can be designed to identify compounds capable of binding said identified gene(s) products of the invention. Compounds identified can be useful, for example, in modulating the activity of wild type and/or mutant gene(s) products, can be useful in elaborating the biological function of said identified gene(s) products, or can disrupt normal identified gene(s) product interactions.

In another aspect the present invention provides a chronic animal model of schizophrenia that mimics the functional deficits observed in patients wherein the animal model has been developed by the addition of PCP to an animal.

In a further aspect the present invention provides a method for developing a chronic animal model of schizophrenia, said method comprising the steps of:

a) administering PCP to an animal in order to induce a psychotic state in the animal representative of the onset of schizophrenia in humans; and

b) further administrating of PCP in order to maintain the PCP-induced psychotic state in the animal, over a period of time, to mimic a chronic state of schizophrenia in the animal.

The present invention also relates to an animal model produced by the method(s) of the present invention.

The animals of the present invention may be any suitable non-human animal, Typically the animal is a rat, mouse, guinea pig, rabbit or the like.

As mentioned above the present invention relates to the development of a chronic animal model. It is understood that the term chronic relates to a disease which is deep-seated or long-continued as opposed to an acute or rapidly developed disease.

The present inventors have developed a chronic treatment paradigm which comprises two phases, The initial phase involves a period of treatment with PCP which was hypothesised would induce a psychotic state within the animal such as a rat, representing the onset of the disease in humans, The second phase concerns the maintenance of this PCP-induced psychotic state over a time period which would allow the incorporation of chronic antipsychotic therapy, relating to the therapeutic delay in antipsychotic efficacy observed in humans. The observation of a psychotic state may be measured in a number of ways.

However, the measurement of the “psychotic state” was determined by the present inventors as PCP-induced hypofrontality which is observed in similar human imaging studies and is correlated to the negative symptoms and cognitive dysfunction associated with chronic schizophrenia (Wolkin, A. et al)

The initial administration of PCP to animal must be sufficient to induce a psychotic state and further administration of PCP must be sufficient to maintain the PCP-induced psychotic state. The present inventors have observed that an initial amount of PCP required to induce a psychotic state may be insufficient to maintain and mimic a chronic state of schizophrenia in the animal.

It has been previously observed that a level of 0.86 mgkg⁻¹ is sufficient to induce an acute state of schizophrenia in an animal model, but the present inventors have found that this is insufficient to maintain and induce a chronic state. The present inventors have used a level of 2.58 mgkg⁻¹ to maintain and induce a chronic state of schizophrenia in a rat model. Thus, the present invention provides a method for developing a chronic animal model of schizophrenia which includes administering a level of 1 to 5 mgkg⁻¹ PCP, for example, a level of 2 to 4 mgkg⁻¹, such as, a level of 2.58 mgkg⁻¹ to an animal to induce a chronic state of schizophrenia.

The effects of this PCP treatment paradigm on dopamine utilisation within selected brain areas was also investigated by HPLC analysis. The levels of dopamine metabolites within plasma and CSF of schizophrenic patients has been established and it has been found that chronic schizophrenics have lower levels of both homovanillic acid (HVA) and dihydroxyphenylacetic acid (DOPAC) compared to controls (Heritch, A, J). This implies that there is decreased turnover of dopamine within the schizophrenic brain.

For a working animal model of a disease to be valid there are certain underlying criteria which are fundamental and which must be taken into consideration. The first criteria, construct validity, is defined as the ability of the model to mimic the underlying neurobiological abnormalities which are core characteristics of the disease. This is difficult to emulate for schizophrenia, since the aetiology of the disease is far from clear. The second criteria, face validity, is defined as the model must produce symptomatologies that resemble those characteristically observed in the disease. The third criteria, predictive validity, is defined as drugs which have established action against a disease must restore parameters in the animal model to normal, whereas other classes of drugs should be inactive.

The chronic PCP model described here satisfies these criteria to an impressive degree. The model uses a drug which is known to produce effects in humans which are analogous to those observed in schizophrenia.

Although the psychotic state may not be triggered by the same mechanism it is likely, from the evidence produced, that the psychosis is being mediated by the same systems which are implicated in the dysfunction associated with schizophrenia, such as the glutamatergic (Tamminga, C.) and dopaminergic (Angrist, B. et al) systems. The model also shows altered function in specific neural circuits, the corticothalamic and temporolimbic circuits, which have been shown to be abnormal in schizophrenia (Swerdlow, N. R. et al and Weinberger, D. R) . The model also has face validity, with metabolic hypofunction, and changes in receptor binding being observed with this model and in schizophrenia. The predictive validity of the model is more difficult to evaluate, although the lack of reversibility of the prefrontal cortex hypofunction mirrors the clinical observations. However, the attenuation of the hypofunction within the auditory system by known antipsychotic drugs suggest that this model does have predictive validity.

The model was also studied for parvalbumin expression which has been shown to be decreased in post mortem tissue of schizophrenic subjects. Parvalbumin expression in the model was also reduced in the prefrontal cortex, as observed in schizophrenic subjects. The model thus reproduces an established pattern of brain dysfunction associated with schizophrenia. This observation may have utility in developing novel antipsychotic drugs.

The model finds particular application in the screening of new drugs for treating schizophrenia. Thus, test drugs may be administered to the animal model and their effect on psychotic conditions observed. The present invention therefore also relates to new anti-schizophrenic drugs identified using the animal model of the present invention.

The model also allows the detection of genes, the expression of which is altered, as compared to a “normal” animal. A “normal” animal is one which has not been induced to the chronic psychotic state and which exhibits normal behaviours.

Genes identified in this manner may be associated with the schizophrenic state. Therefore identification of such genes allows their study and/or development of therapies designed to return expression to normal.

The present invention will now be further described by way of non-limiting example and with reference to the attached Figures (where CLO indicated clozapine and HAL indicates haloperidol) which show:

FIGS. 1-4 show the nucleotide sequence of four sequences observed to be differentially expressed in the brain of the rat model of the present invention;

FIG. 5a shows the nucleotide sequence and FIG. 5b shows the polypeptide sequence of phosphodiesterase 1α which have been observed to be differentially expressed in the brain of the rat model of the present invention;

FIGS. 6a, 6c and 6e show the nucleotide sequences and FIGS. 6b, 6d and 6f show the polypeptide sequences of calcium-independent alpha-latrotoxin receptor which have been observed to be differentially expressed in the brain of the rat model of the present invention;

FIG. 7a shows the nucleotide sequence and FIG. 7b shows the polypeptide sequence of epithelial discoidin domain receptor, trkE, which has been observed to be differentially expressed in the blood of schizophrenic patients as compared to normal controls;

FIG. 8a shows the nucleotide sequence and FIG. 8b shows the polypeptide sequence of netrin receptor which has been observed to be differentially expressed in the brain of the rat model of the present invention;

FIGS. 9a and 9c show the nucleotide sequence and FIGS. 9b and 9d show the polypeptide sequence of synapsins 1A and 1B which have been observed to be differentially expressed in the brain of the rat model of the present invention;

FIG. 10a shows the nucleotide sequence and FIG. 10b shows the polypeptide sequence of YSG9 (SEQ ID No. 19) which has been observed to be differentially expressed in the brains of schizophrenic patients and PCP-treated rats as compared to normal controls;

FIG. 11 is a histogram showing the relative expression levels of genes in human blood samples;

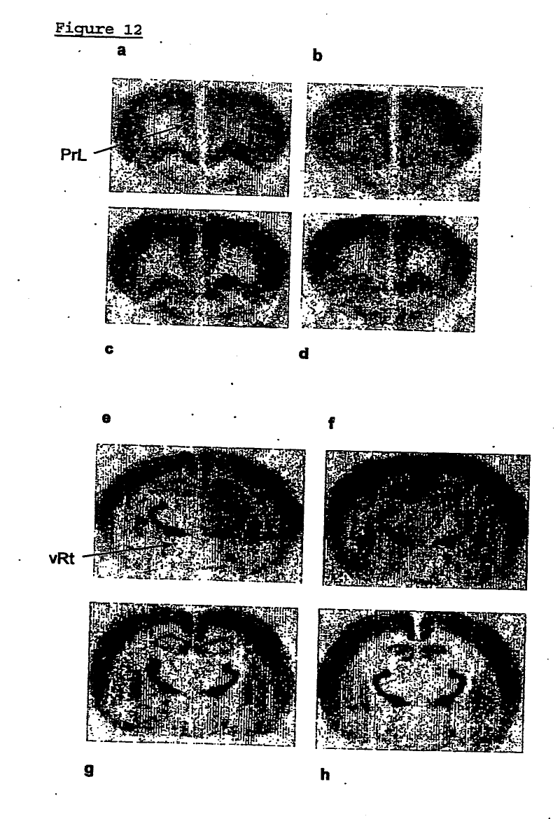

FIG. 12 shows parvalbumin expression in brain tissue of the animal model of the present invention;

FIG. 13 illustrates the level of CIRL1 mRNA present in the BA11 region of schizophrenic (grey dashed line, n=6) and control (black, n=8) post-mortem tissue. TCTCCTGGCTGTGCCTGGAGGGC and GGCTTGAGCACAGATCAGCTTCGG (SEQ ID NOS: 23-24) were the primer sequences used to amplify this product.

FIG. 14 illustrates the level of CIRL1 mRNA present in the prefrontal cortex of rat brain following chronic PCP and antipsychotic treatment (n=12 for all groups apart from veh-veh where n=11). TCTCCTGGCTGTGCCTAGAGGGC and GGCTTGAGCACGGATGAGCTTCGG (SEQ ID NOS: 25-26) were the primer sequences used to amplify this product.

FIG. 15 illustrates the level of CIRL2 variant AB mRNA present in the prefrontal cortex of rat brain following chronic PCP and antipsychotic treatment (n=12 for all groups apart from veh-veh where n=11). GGAAAACATTAAGTCTTGGGTG and GTGAATGTCCTTGATTAAGGGT (SEQ ID NOS: 27-28) were the primer sequences used to amplify this product.

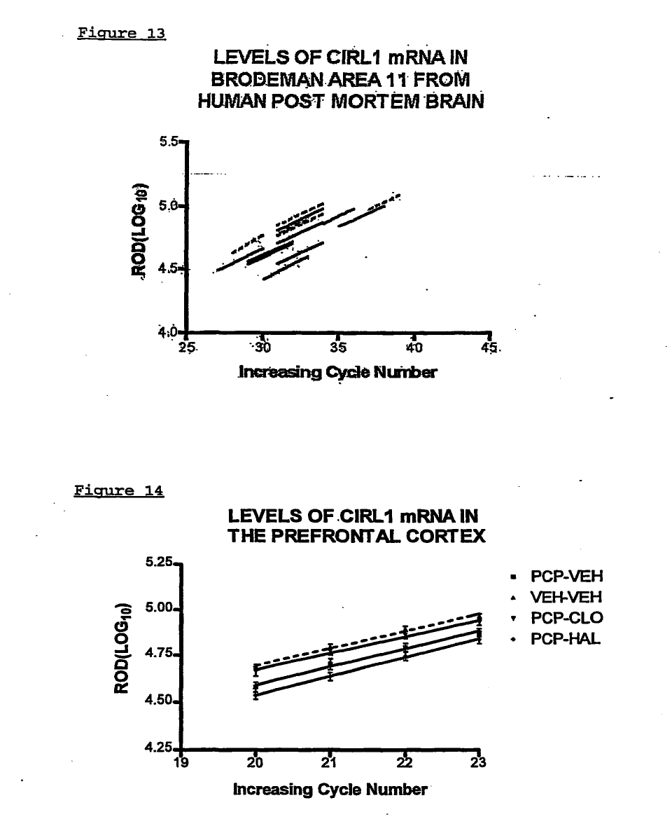

FIG. 16 illustrates the level of CIRL3 variant AA mRNA present in the prefrontal cortex of rat brain following chronic PCP and antipsychotic treatment (n=12 for all groups apart from veh-veh where n=11) GTAGTTCATGCTTTCAGCCGT and AGAAGCCCCTCTCTGTTGAG (SEQ ID NOS: 29-30) were the primer sequences used to amplify this product.

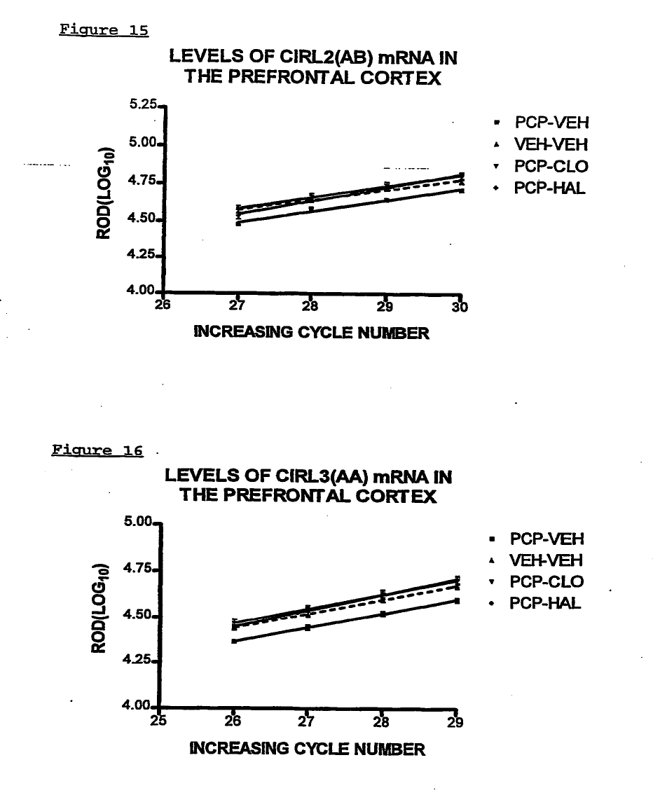

FIG. 17 illustrates the expression profile of TNFα 2 and 24 hrs after a single i.p. injection of PCP at 2 mg/kg (N=4 for all treatment groups). AGGAGGAGAAGTTCCCAAATG and TTGTCCCTTGAAGAGAACCTG (SEQ ID NOS: 31-32) were the primer sequences used to amplify this product.

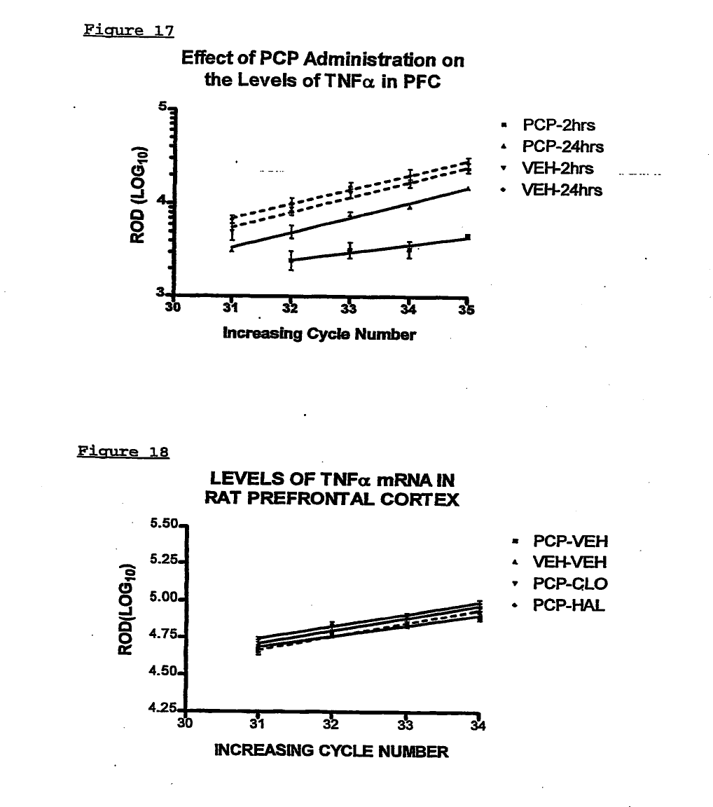

FIG. 18 illustrates the levels of TNFα in rat prefrontal cortex following chronic PCP and antipsychotic treatment (n=12 for all groups apart from veh-veh where n=11). AGGAGGAGAAGTTCCCAAATG and TTGTCCCTTGAAGAGAACCTG (SEQ ID NOS: 31-32) were the primer sequences used to amplify this product.

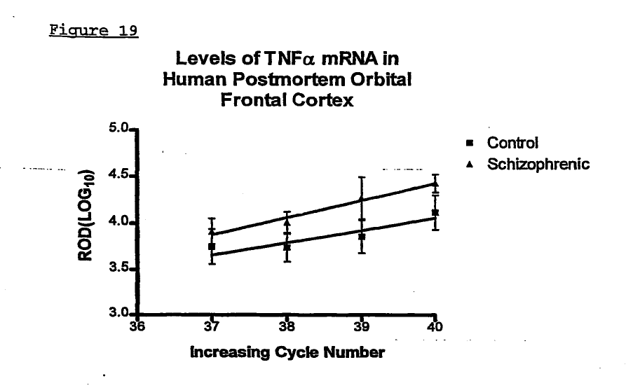

FIG. 19 illustrates the levels of TNFα in postmortem orbital frontal cortex of schizophrenics (n=4) and controls (n=5). GGTAGGAGACGGCGATGC and CAGGCAGTCAGATCATCTTC (SEQ ID NOS: 33-34) were the primer sequences used to amplify this product.

### Example 1

Development of Rat Model

An initial treatment period of intraperitoneal (i.p.) injections once daily for 5 days was carried out, followed by a maintenance schedule of i.p. injections three times weekly (every 60 hours) for a further 21 days.

Intermittent exposure to PCP during the maintenance phase of the model was favoured due to the long half life of the drug within brain tissue (Misra, A. L. et al). The doses of PCP chosen represented the selective blockade of the NMDA channel (0.86 mgkg⁻¹) and a dose (2.58 mgkg⁻¹) which is pharmacologically less selective but less that the ED₅₀ for PCP-induced cell death. As a comparison to the present model, the inventors also investigated the effect of previously published subchronic treatment with PCP (Jentsch, J. D. et al) using quantitative C-2-deoxyglucose autoradiography (Sokoloff, L.)

Local cerebral glucose utilisation (LCGU) was measured using an adaptation of the original method for freely moving rats (Crane, A. M. et al) 72 hours after the initial induction phase (day 8) and 72 hours after the induction phase followed by the maintenance phase (day 29). LCGU was measured 72 hours after the last exposure to PCP so the effects of PCP on LCGU would be independent of the acute effects of the drug. Table 1 shows the results from the induction and maintenance phases of the model. The dose of 2.58 mgkg⁻¹ PCP induced a metabolic hypofunction which was evident after both phases of the model within the medial orbital cortex, the prelimbic cortex, the auditory pathway and the reticular nucleus of the thalamus. The metabolic hypofunction produced by the lower dose of PCP (0.86 mgkg⁻¹) within these areas during the initial phase of the model was not, however, maintained by the subsequent second phase of the model. Thus, using a dose of 2.59 mgkg⁻¹ the inventors had established a novel treatment paradigm which mimics the findings of human imaging studies in schizophrenic patients. In comparison, the previously published subchronic treatment (Jentsch et al) with PCP (5 mgkg⁻¹ twice daily for seven days) did not produce any significant effect on LCGU within any brain area (data not shown)

In summary, the data provided from the animal studies utilising this chronic PCP model mimic those previously published from human imaging studies and post mortem studies of schizophrenic brain tissue from schizophrenic patients. Since human imaging studies have correlated the prefrontal hypofunction to the negative symptoms of schizophrenia (Wolkin, A. et al, 1992) and abnormalities of the temperolimbic system (including the auditory system and hippocampus) and thalamus to the positive symptoms of schizophrenia (Tamminga, C. A. et al, 1992), it can be proposed that this model mimics both the positive and negative symptoms of the disease. As such, this model has superior construct, face and predictive validity than existing animal models of schizophrenia.

TABLE 1
Induction and maintenance of PCP-induced hypofunction

| | LCGU (μmol/100 g/min) | | | | |
|---|---|---|---|---|---|
| day 8 | | | day 29 | | |
| | 0.86 | 2.58 | | 0.86 | 2.58 |
| vehicle | PCP | PCP | vehicle | PCP | PCP |

Prefrontal Cortex

| | | | | | | |
|---|---|---|---|---|---|---|
| mO layer | 131 ± 4 | 110 ± 2* | 105 ± 4* | 125 ± 4 | 122 ± 3 | 108 ± 5* |
| mO layers | 137 ± 4 | 127 ± 2 | 127 ± 5 | 147 ± 3 | 135 ± 4 | 124 ± 5 |
| II & III | | | | | | |
| mO layers | 140 ± 1 | 129 ± 5* | 115 ± 1* | 137 ± 2 | 136 ± 3 | 111 ± 3* |
| V & VI | | | | | | |
| PrL layer I | 134 ± 2 | 132 ± 6 | 104 ± 2* | 135 ± 1 | 133 ± 2 | 107 ± 4* |
| PrL layers II | 154 ± 3 | 150 ± 7 | 133 ± 2* | 152 ± 2 | 149 ± 2 | 116 ± 1* |
| & III | | | | | | |
| PrL layers | 114 ± 3 | 115 ± 3 | 96 ± 3* | 114 ± 2 | 112 ± 3 | 89 ± 2* |
| V & VI | | | | | | |

Thalamus

| | | | | | | |
|---|---|---|---|---|---|---|
| Rt | 116 ± 2 | 106 ± 2 | 86 ± 2* | 118 ± 4 | 108 ± 4 | 89 ± 2* |
| MD | 114 ± 4 | 112 ± 4 | 115 ± 4 | 121 ± 6 | 122 ± 5 | 116 ± 3 |

Auditory System

| | | | | | | |
|---|---|---|---|---|---|---|
| Au layer I | 159 ± 11 | 127 ± 2 | 135 ± 3* | 158 ± 13 | 167 ± 14 | 137 ± 5* |
| Au layers | 171 ± 13 | 152 ± 3 | 166 ± 5 | 184 ± 8 | 189 ± 12 | 162 ± 6 |
| II, III&IV | | | | | | |
| Au layers V | 122 ± 8 | 114 ± 4 | 120 ± 4 | 128 ± 9 | 137 ± 10 | 115 ± 3 |
| & VI | | | | | | |
| AuD layer I | 155 ± 12 | 126 ± 3* | 135 ± 3* | 167 ± 9 | 159 ± 11 | 130 ± 7* |
| AuD layers | 178 ± 14 | 151 ± 4* | 151 ± 4* | 189 ± 9 | 178 ± 10 | 146 ± 7* |
| II, III&IV | | | | | | |
| AuD layers | 127 ± 7 | 106 ± 2* | 104 ± 2* | 133 ± 7 | 128 ± 9 | 101 ± 4* |
| V & VI | | | | | | |
| DLL | 116 ± 7 | 91 ± 4* | 83 ± 4* | 118 ± 7 | 116 ± 8 | 96 ± 5* |
| VLL | 125 ± 9 | 98 ± 5* | 93 ± 3* | 121 ± 6 | 118 ± 13 | 99 ± 4* |
| cochlear | 126 ± 4 | 107 ± 3* | 98 ± 3* | 124 ± 3 | 117 ± 2 | 94 ± 4* |
| nucleus | | | | | | |

Table 1: All data expressed as mean LCGU (μmol/100 g/min) ± SEM (n = 5-6).
Statistical analysis carried out using individual one-way ANOVA for each discrete brain region followed by Fisher's least significant difference post hoc test where appropriate, with statistical significance defined as p < 0.05.
*p < 0.05 compared to controls.
Day 8 data represents LCGU measured 72 hours following the last exposure to PCP after 5 days i.p. injections once daily of 0.86 or 2.58 mgkg⁻¹ PCP or vehicle (sterile saline).
Day 29 data represents LCGU measured 72 hours following the last exposure to PCP after i.p. injections once daily (day 1-5) and once daily on days 8, 10, 12, 15, 17, 19, 22, 24 and 26 of 0.86 or 2.58 mgkg⁻¹ PCP or vehicle (sterile saline).
Abbreviations:
mO, medial orbital cortex;
PrL, prelimibic cortex;
Rt reticular nucleus of the thalamus;
MD, mediodorsal nucleus of the thalamus;
Au, primary auditory cortex;
AuD, dorsal nucleus of the secondary auditory cortex;
DLL & VLL, dorsal nucleus and ventral nucleus of the lateral lemniscus.

### Example 2

Testing of Rat Model

In order to establish the effect of antipsychotic drugs in the model, a second study was then carried out using a dose of 2.58 mgkg¹ PCP which produced a metabolic hypofunction in the first studies, combined with antipsychotic therapy. The antipsychotic drugs were administered via osmotic minipumps for 21 days in order to maintain constant plasma concentrations of the drugs, which mirrored therapeutic plasma levels of the drugs in humans.

Table 2 shows the effect of haloperidol and clozapine alone and in conjunction with PCP treatment compared to vehicle treated rats. Within the medial orbital cortex, the prelimbic cortex, the CA1 region of the hippocampus and the reticular nucleus of the thalamus, a metabolic hypofunction was again observed after treatment with PCP compared to controls. Clozapine and haloperidol also produced a metabolic hypofunction within these areas and failed to modulate the hypofunction produced by PCP.

Within the auditory system, the dorsal nucleus of the secondary auditory cortex, dorsal and lateral nucleic of the lateral lemniscus and the cochlear nucleus, PCP again induced a metabolic hypofunction. However, within these regions, clozapine and haloperidol did not produce a significant hypofunction by themselves, but reversed the PCP-induced hypofunction when used in conjunction with the PCP. The inability of haloperidol and clozapine to modulate the hypofrontality is consistent with data from clinical studies and also the theory that this hypofrontality is associated with the negative symptoms and cognitive dysfunction of schizophrenia. The effect of antipsychotics on the positive symptoms is less well studied regarding imaging studies. There is no published evidence to date regarding the effect of haloperidol and clozapine within the temporal lobe structures (hippocampus and auditory cortex).

However, the ability of both antipsychotics to reverse the decreased glucose utilisation within the auditory system (auditory cortex, lateral lemniscus and the cochlear nucleus) is consistent with the clinical evidence that both typical and atypical antipsychotics can improve ratings of positive symptoms of schizophrenia.

In order to further validate this chronic PCP model, the effects of this treatment paradigm on 5-HT_{2A} receptors within the prefrontal cortex was investigated. Chronic PCP treatment produced a significant decrease in 5-HT_{2A} receptors in layer II & III (controls 158±6, PCP 139±4 fmolmg⁻¹) and layers V & VI (controls 82±4, PCP 69±3 fmolmg⁻¹). This is entirely consistent with post mortem studies of 5-HT_{2A} receptor binding from schizophrenic patents (Laurelle, M. et al).

In order to validate further this chronic PCP model the effect of this treatment paradigm on parvalbumin mRNA expression was investigated. A decrease in parvalbumin mRNA was observed after chronic PCP treatment within the prelimbic region of the prefrontal cortex (controls 0.0717±0.0011, PCP 0.0536±0.0023 relative optical density (ROD)). This PCP-induced decrease was reversed by clozapine (0.0693±0.0050 ROD) but not by haloperidol (0.0557±0.0022 ROD) . PCP produced a significant decrease in parvalbumin mRNA within the ventral reticular nucleus of the thalamus (controls 0.6416±0.0122, PCP 0.5032±0.0194 ROD) which was reversed by both clozapine (0.6354±0.0173 ROD) and haloperidol (0.06199±0.0137) (see FIG. 12). This decrease in parvalbumin expression is in agreement with studies of schizophrenic post mortem tissue within the prefrontal cortex (Beasley & Reynolds, 1997) and anterior thalamus (Danos et al, 1998). The ability of clozapine but not haloperidol to reverse the decrease in parvalbumin expression in the prefrontal cortex is consistent with its ability to alleviate the cognitive deficits/negative symptoms in schizophrenia. Thus, reversal of parvalbumin deficits may be a useful marker for detecting atypical antipsychotic activity.

Methods

5-HT_{2A} receptor binding: Sections from the level of the prefrontal cortex were preincubated for two consecutive washes at room temperature in 50mM Tris HCl buffer pH 7.4 to remove endogenous ligand. Total binding was defined using 0.71 nM (Wolkin, A. et al) ³H-ketanserin in the presence of 1μM prazozin and 1μM tetrabenazine (to block non 5-HT_{2A} binding). Non-specific binding was defined using 50nM spiperone. Sections were incubated with the appropriate ligand solution for 1 hour at room temperature then washed twice for 10 minutes in ice cold buffer before being rinsed in ice cold water and rapidly air dried. The sections were then exposed to film (Biomax MR, Kodak) with previously calibrated (Wolkin, A. et al) ³H-standards.

Autoradiograms were analysed using MCID densitometry system. Results were statistically analysed using a one-way ANOVA followed by a student Newman-Keuls post hoc test.

In situ hybridisation: a 45mer oligonucleotide probe was designed against bases 223-267 of the rat parvalbumin gene (GenBank accession number A819345). In situ hybridisation was carried out according to the method of Wisden and Morris (1994).

TABLE 2
Effect of haloperidol and clozapine on PCP induced hypofunction

| LCGU (μmol/100 g/min) | | | | | |
|---|---|---|---|---|---|
| vehicle | | | PCP | | |
| vehicle | Clz | hal | vehicle | Clz | hal |

Prefrontal Cortex

| | | | | | | |
|---|---|---|---|---|---|---|
| mO layer I | 127 ± 7 | 93 ± 4* | 93 ± 4* | 104 ± 5* | 104 ± 5* | 105 ± 4 |
| mO layers | 138 ± 6 | 109 ± 4* | 106 ± 4* | 121 ± 6 | 112 ± 6* | 116 ± 4* |
| II & III | | | | | | |
| mO layers | 135 ± 9 | 106 ± 5* | 102 ± 4* | 113 ± 6 | 115 ± 5* | 119 ± 4 |
| V & VI | | | | | | |
| PrL layer I | 139 ± 5 | 119 ± 4* | 114 ± 4* | 109 ± 4* | 118 ± 6* | 115 ± 3* |
| PrL layers II | 152 ± 7 | 139 ± 5 | 131 ± 4 | 127 ± 5* | 134 ± 7 | 134 ± 4 |
| & III | | | | | | |
| PrL layers | 116 ± 5 | 98 ± 4* | 93 ± 4* | 97 ± 3* | 104 ± 5 | 97 ± 3* |
| V & VI | | | | | | |

Thalamus

| | | | | | | |
|---|---|---|---|---|---|---|
| Rt | 112 ± 6 | 95 ± 4* | 86 ± 4* | 79 ± 2* | 81 ± 2* | 80 ± 1* |
| MD | 130 ± 6 | 124 ± 6 | 117 ± 5 | 133 ± 6 | 113 ± 3 | 119 ± 6 |

Auditory System

| | | | | | | |
|---|---|---|---|---|---|---|
| Au layer I | 153 ± 5 | 136 ± 6 | 142 ± 9 | 138 ± 3 | 140 ± 5 | 141 ± 8 |
| Au layers | 183 ± 8 | 169 ± 6 | 167 ± 9 | 174 ± 4 | 166 ± 6 | 174 ± 10 |
| II, III&IV | | | | | | |
| Au layers V | 126 ± 2 | 126 ± 4 | 112 ± 9 | 120 ± 2 | 118 ± 4 | 117 ± 7 |
| & VI | | | | | | |
| AuD layer I | 157 ± 8 | 136 ± 4 | 139 ± 9 | 126 ± 5* | 135 ± 5 | 133 ± 7 |
| AuD layers | 170 ± 10 | 154 ± 5 | 153 ± 9 | 141 ± 6* | 152 ± 6 | 156 ± 10 |
| II, III&IV | | | | | | |
| AuD layers | 122 ± 2 | 115 ± 6 | 108 ± 7 | 105 ± 2* | 107 ± 4 | 106 ± 6* |
| V & VI | | | | | | |
| DLL | 112 ± 5 | 99 ± 5 | 92 ± 4 | 89 ± 4* | 108 ± 5 | 107 ± 2 |
| VLL | 120 ± 4 | 109 ± 5 | 106 ± 5 | 98 ± 6* | 118 ± 5 | 110 ± 3 |
| cochlear | 125 ± 2 | 108 ± 7 | 99 ± 9* | 92 ± 4* | 119 ± 8 | 115 ± 2 |
| nucleus | | | | | | |

Hippocampus

| | | | | | | |
|---|---|---|---|---|---|---|
| CA1 molecular | 106 ± 3 | 102 ± 5 | 92 ± 5 | 93 ± 2 | 87 ± 2* | 97 ± 4 |
| layer | | | | | | |
| CA1 stratum | 82 ± 3 | 80 ± 4 | 66 ± 4* | 67 ± 2* | 66 ± 3* | 74 ± 4 |
| radiatum | | | | | | |
| CA1 pyramidal | 79 ± 3 | 78 ± 4 | 63 ± 4* | 63 ± 2* | 62 ± 2* | 70 ± 4 |
| cell layer | | | | | | |
| CA1 stratum | 73 ± 3 | 72 ± 4 | 59 ± 4* | 59 ± 2* | 57 ± 2* | 64 ± 4 |
| oriens | | | | | | |
| CA3 molecular | 96 ± 2 | 96 ± 4 | 90 ± 4 | 90 ± 2 | 84 ± 2 | 94 ± 6 |
| layer | | | | | | |
| CA3 stratum | 76 ± 2 | 83 ± 5 | 73 ± 1 | 70 ± 3 | 69 ± 2 | 76 ± 5 |
| radiatum | | | | | | |
| CA3 pyramidal | 75 ± 3 | 81 ± 4 | 71 ± 4 | 69 ± 2 | 69 ± 3 | 74 ± 5 |
| cell layer | | | | | | |
| CA3 stratum | 69 ± 3 | 74 ± 4 | 64 ± 4 | 60 ± 3 | 62 ± 1 | 69 ± 5 |
| oriens | | | | | | |

Table 2:
All data expressed as mean LCGU (μmol/100 g/min) ± SEM (n = 6).
Statistical analysis carried out using individual two-way ANOVA for each discrete brain region followed by Tukey's post hoc test where appropriate, with statistical significant defined as p < 0.05.
*p < 0.05 compared to controls.
The treatment paradigm was as follows: once daily i.p. injections of PCP (2.58 mgkg⁻¹) or vehicle (saline) on days 1 to 5 (phase 1), implantation of primed osmotic minipumps on day 8 (vehicle, clozapine 20 mgkg⁻¹/day, haloperidol 1 mgkg⁻¹/day), i.p. injections of PCP or vehicle once daily on days 8, 10, 12, 15, 17, 19, 22, 24 and 26 (phase 2) with the animals killed on day 29.
Abbreviations are as in Table 1 legend.

### Example 3

Use of PCP Model to Discover Novel Genes Potentially Important in Schizophrenia and its Treatment

The PCP model as described herein has been used to identify novel genes for schizophrenia using two different molecular biology approaches.

1) Atlas Arrays

Four groups of rats were treated with (a) chronic PCP, (see Example 1), (b) chronic vehicle (control), (c) chronic PCP plus chronic clozapine and (d) chronic PCP plus chronic haloperidol.

Rats were injected with PCP (2.58 mg/kg) or vehicle i.p. for 5 days according to the YRING PCP model. On day 7, they were implanted with osmotic minipumps containing either clozapine or haloperidol at concentrations that would administer drugs at 20 or 1 mg/kg/day respectively, or vehicle. On the same day, the rats began a course of i.p. injections every 2.5 days with either PCP (2.58 mg/kg) or vehicle. This regimen gave the following treatment groups:

| I.P. | Minipump | N^{o} |
|---|---|---|
| Vehicle | Vehicle | 6 |
| PCP | Vehicle | 6 |
| PCP | Clozapine | 6 |
| PCP | Haloperidol | 6 |

21 days after minipump implantation, animals were killed by cervical dislocation and the prefrontal cortex dissected and stored at −70° C. RNA was then prepared according to the protocol below and the corresponding cDNA synthesis and hybridisation procedure were conducted using the rat Atlas Array kit according to the manufacturer's instructions (Clontech). Several genes were affected by the treatments. Of particular interest was E3C (calcium independent alpha-latrotoxin receptor CIRL) which showed an increase after the PeP treatment regime and which was reversed by the antipsychotic drugs haloperidol and clozapine. A second experiment has been performed using the same treatment regimes with an n=4 per group (each value being pooled prefrontal cortex tissue from 3 rats).

Significant increases in CIRL were confirmed after chronic PCP and in addition there were significant increases in expression of UNC5H1 (a netrin receptor) and synapsins (1A and 1B) after chronic PCP as compared to the vehicle treated control group (see Table below)

| | Vehicle | | Significance; |
|---|---|---|---|
| Gene | control | Chronic PCP | t test |
| CIRL-1 | 4542 ± 804 | 9145 ± 669 | P < 0.009 |
| UNC5H1 | 1410 ± 480 | 3936 ± 472 | P < 0.015 |
| Synapsins | 17365 ± 1144 | 23020 ± 1412 | P < 0.025 |
| 1A&1B | | | |

Results are expressed as mean relative optical densities ±SEM. Statistical significance was defined as P<0.05. N=¾ per group.

Protocol for RNA Preparation for Atlas Arrays

Frozen tissue already resides in the ribolyser tubes from the dissection procedure
1) Add 1.1 ml Qiagen lysis buffer (containing β-mercaptoethanol, final volume =3%).
2) Perform 3×20 sec homogenisations at 6.5 g in a ribolyser.
3) Spin 3 min in microfuge.
4) Decant to fresh tube and re-spin 3 min.
5) Decide at this stage if you want to dilute supernatant with more lysis buffer.
6) Add equal volume of phenol/CHCl₃ pH4.7, vortex 30 sec and leave on ice for 10 min.
7) Re-vortex and spin 4° C. 5-10 min at 1500 g.
8) Decant supernatant and add 100 μl H₂O. Add an equal volume of phenol/CHCl₃ pH4.7, vortex, spin 5-10 min at 1500 g.
9) Decant supernatant and add 100 μl H₂O. Add an equal volume of CHCl₃, vortex, spin 5-10 min at 1500 g.
10) Decant supernatant to fresh tube.
11) Re-extract with more lysis buffer and proceed through steps 6-9 and pool fraction with stage 10 (do not add H₂O to supernatants).
12) Measure supernatant volume, add 0.1 vol. 2M NaOAC and 2.5 vol. (total vol.) ethanol, mix and leave at −80° C. at least 1 hr.
13) Spin 1500 g for 15-20 min at 4° C.
14) Wash pellet with 70% EtOH.
15) Spin 5 min.
16) Decant supernatant, quick spin, remove rest of supernatant with a pipette.
17) Air dry pellet (don't over dry).
18) Re-suspend pellet in 60μl H₂O.
19) Measure 0D₂₆₀.
20) DNase 1 treat RNA according to the MessageClean (Genhunter) protocol (except perform additional re-extraction with H₂O)
21) Re-suspend in as little H₂O as possible (12 μl) to keep the RNA concentrated for the Atlas cDNA synthesis step.
22) 20μg of RNA in a final volume of 5μl is used to generate cDNA according to protocols outlined in the Atlas Array manual.
   2) Further Verification of the Importance of CIRL

Samples of human schizophrenic brain and age matched control tissue (obtained from Professor G Reynolds, University of Sheffield) were examined by RT-PCR for changes in the expression of CIRL.

In addition, four groups of rats were treated with a) chronic PCP, chronic vehicle (control), c) chronic PCP plus clozapine and d) chronic PCP plus chronic haloperidol as detailed previously in Atlas Array experiment (p.32). RT-PCR for specific isoforms of CIRL was then conducted in the prefrontal cortex.

Method for Brain Tissue Preparation (Rat and Human) RT-PCR Protocol

Isolation of Total RNA

1 ml of lysis buffer (Qiagen), including 1% β-mercaptoethanol, was added to approximately 50-100 mg of brain tissue. Tissue samples were homogenised using a ribolyser (Hybaid) with 3 bursts at 6.5 g lasting 20 seconds each. Samples were then spun at room temperature in a microfuge for 3 minutes. The supernatant was removed and re-spun for a further 3 minutes. The supernatant was decanted to a fresh tube to which an equal volume of 70% ethanol was added. The remaining RNA isolation procedure was carried out according to the manufacturer's protocol (Qiagen).

Synthesis of cDNA

Synthesis of cDNA was carried out according to manufacturers protocols (Life Technologies). Briefly 3-5μg of total RNA was reverse transcribed using oligo dT priming. After cDNA synthesis, samples were aliquoted and stored at −70° C. The amount of cDNA in each aliquot would allow a PCR titration at four different cycles with an input RNA template concentration of about 75 ng for each PCR reaction

PCR

Alterations in expression levels were determined by semi-quantitative PCR. Expression levels between different samples were standardised against the amount of β-actin mRNA present in each sample. Briefly, known amounts of template were PCR amplified. Samples were removed over 4 consecutive cycles, however, the first cycle to be removed sometimes varied depending on when logarithmic amplification was detected. Samples were separated on agarose gels and stained with GelStar solution (Flowgen). Results were plotted as the log₁₀ of relative optical density of bands against increasing cycle number. Linear regression analysis was performed. For β-actin titrations, values were obtained from the intersection of the regression lines with the Y-axis. These values were standardised against a single sample. Standardisation coefficients generated at this step were used to standardise the data from target gene expression levels.

Results

The levels of CIRL1 mRNA increased in Brodman Area 11 in postmortem schizophrenic brain tissues as compared to controls suggesting that alterations in CIRL may be important in the schizophrenic disease state (see FIG. 13).

Analysis of selected specific isoforms of CIRL in rat brain revealed that chronic PCP treatment reduced the expression of CIRL1, CIRL2 (AB) and CIRL3 (AA) in the prefrontal cortex (see FIGS. 14, 15 and 16) . There was a reversal of the PCP-induced reductions in the level of CIRL1 mRNA by the atypical antipsychotic drug clozapine but not by the typical antipsychotic drug haloperidol. Both drugs reversed the PCP-induced reductions in CIRL2 and CIRL3.

These data support CIRL as a therapeutic target for antipsychotic drug activity.

3) Differential Display

Four groups of rats were treated with (a) chronic PCP, (b) chronic vehicle (control), (c) chronic PCP plus chronic clozapine (d) chronic vehicle plus chronmc clozapine (as above).

Differential display was performed according to the method of Liang and Pardee (Molecular Biotechnology, 110, 261-267, 1998). Prefrontal cortex tissue was dissected, and total RNA extracted using Qiagen's “RNeasy” kit. An oligo(dT) primer was then used for cDNA synthesis usmng MMLV reverse transcriptase. The cDNA template obtained was used as a basins for the polymerase chain reaction (PCR) using the Clontech “Delta” differential display kit.

Various pairwise combinations of arbitrary primers and “Advantage 2” polymerase were employed according to the Clontech “Delta” differential display kit manual. Differential display products were electrophoresed on 6% acrylamide gels and exposed to x-ray film. Bands corresponding to cDNA fragments differentially expressed between prefrontal cortex tissue from vehicle-treated animals and PCP-treated animals were excised, and reamplified using the original primers. Differential expression was then confirmed using further prefrontal cortex tissue from these treatment groups. The cDNAs with verified differential expression were sub-cloned and sequenced, and the sequence information obtained subsequently compared with the “DNA Data Bank of Japan” database, for homology with known genes or ESTs.

Three novel sequences were identified (SEQ ID Nos. 1, 2, 3 and 4) as being differentially expressed as well as the previously known gene for phosphodiaesterase 1α. Further confirmation of changes in expression of the above identified nucleotide sequences was confirmed following chronic PCP treatment of the rat model by semi-quantitative RT-PCR (data not shown).

4) RT-PCR

Four groups of rats were treated with (a) chronic PCP, (b) chronic vehicle (control), (c) chronic PCP plus chronic clozapine (as above) or (d) chronic PCP plus chronic haloperidol (as above). The tissue was processed for RNA extraction and RT-PCP as described above, using primers specific for TNFα mRNA.

Acute PCP treatment reduced the levels of TNFα in rat prefrontal cortex (see FIG. 17). This effect was apparent 2 hrs and 24 hrs following drug treatment.

In groups of rats chronically treated with PCP and antipsychotic drugs (see p.32 for details), PCP in combination with haloperidol was significantly different from the chronic PCP group (see FIG. 18)

Further studies revealed a significant increase in TNFα mRNA levels in the Orbital frontal cortex of schizophrenic patents (see FIG. 19).

These results implicate TNFα in the development and treatment of schizophrenia.

### Example 4

Differentially Expressed Genes in Human Blood Samples using cDNA Macroarrays

Materials and Methods

Human male blood samples from schizophrenics and healthy volunteers were obtained from Gartnavel Royal Hospital, Glasgow, UK, with consent. The profile of samples are shown in Table 3.

Total RNAs were isolated from human bloods using TRIzol LS Reagent (Gibco/BRL) and treated with DNase I. Four to 8 μg of total RNAs were used as templates for cDNAs. ³³P radiolabelled cDNAs were hybridised with the Atlas™ Human Cytokine/Receptor Arrays (Clontech). The arrays were washed and then exposed to X-ray films. The spots on the films were analysed by densitometry. Data were analysed using independent samples t-test. Statistical significance was defined as p<0.05.

Results and Discussion

In this study, only 24 to 93 out of 268 genes could be measured. This could be due to several reasons. Firstly, many cytokines are poorly expressed. Secondly, the efficiency of 1^{st} strand cDNA synthesis could have been low due to usage of total RNA instead of mRNA. Because of the limited amount of samples available, total RNA was utilised. Finally, some membranes had extremely high background which could not be washed out even boiling the membranes.

At first, the expression levels of genes were compared to each of 3 housekeeping genes, ubiquitin, ribosomal protein S9 and phospholipase A2 for the purpose to correcting the amount of input RNA. Slightly different results were obtained when different housekeeping genes were used to standardise signals. So each relative expression level from 3 housekeeping genes was averaged for lowering the deviation. Only epithelial discoidin domain receptor 1, trkE (23 j) showed significant difference between schizophrenics and controls (see FIG. 12). This kinase is purported to be a receptor for nerve growth factor and expressed at low levels in most tissues and expression is highest in the brain and lung (Perez et al).

A recent paper showed that trkC mRNA levels in schizophrenics were decreased in the frontal cortex (Schramm et al). TrkC is a high-affinity receptor for neurotrophin-3. Neurotrophins and their receptors have been implicated in the molecular-pathology in schizophrenia (Bayer & Falkai). TrkE might also show the reduction with trkC.

TABLE 3
Profile of human blood samples
Schizophrenics

| Code No. | Smoker | Age | Weight (kg) | Medication Medical History |
|---|---|---|---|---|
| 01 | No | 30 | 89 | Clz 500 mg/day 14 yr |
| 02 | No | 40 | 98 | Clz 250 mg/day 20 yr |
| 25 | Yes | 55 | 70 | Clz 250 mg/day 20 yr |
| 27 | No | 42 | 103 | clz 600 mg/day 24 yr |
| | | | | Fpz 75 mg/2weeks |
| 34 | No | 46 | 80 | Clz 100 mg/day 23 yr |
| | | | | Diclofenac sodium |

42.6 ± 9.1

Controls

| Code | | | | | |
|---|---|---|---|---|---|
| No | Smoker | Age | Weight (kg) | Medication | Medical History |
| 04 | Yes | 32 | 76 | — | — |
| 24 | No | 44 | 95 | — | — |
| 28 | No | 27 | 73 | — | — |
| 32 | No | 35 | 84 | — | Sore Throat |
| 35 | No | 37 | 70 | CoProxamol | — |

35.0 ± 6.3

### REFERENCES

1. Goldberg, T. et al, The impact of clozapine on cognitive impairment and psychiatric symptomatology in patients with schizophrenia. Br. J. Psychiatry 162, 43-48 (1993).
2. Allen, R. M. & Young, S. J. Phencyclidine-induced psychosis. Am. J. Psychiatry 135, 1081-1084 (1978).
3. Wolkin, A. et al. Persistence of cerebral metabolic abnormalities in chronic schizophrenia as determined by positron emission tomography. Am. J. Psychiatry 142, 564-571 (1985).
4. Misra, A. L., Pontani, R. B. & Bartolomeo, J. G. Disposition of [³H] phencyclidine in the rat after single and multiple doses. Life Sci, 27, 2501-2508 (1980).
5. Jentsch, J. D., Tran, A, Le, D, Toungren, K. D. & Roth, R. H. Subchronic phencyclidine administration reduces mesolimbic prefrontal dopamine utilisation and impairs prefrontal cortical-dependent cognition in the rat. Neuropsychopharm. 17, 92-99 (1997).
6. Sokoloff, L. The 14-C-deoxyglucose method for the measurement of local cerebral glucose utilisation: theory, procedure and normal values in the conscious and anaesthetised albino rat, J. Neurochem. 28, 897-916 (1977).
7. Crane, A. M. & Porrino, L. J. Adaptation of the quantitative 2-[14-C]-deoxyglucose method for use in freely moving rats. Brain Res. 499, 87-92 (1989).
8. Laurelle, M. et al. Selective abnormalities of prefrontal serotonergic receptors in schizophrenia, a postmortem study. Arch. Gen. Psychiatry 50, 810-818 (1993).
9. Heritch, A. J. Evidence for reduced and dysregulated turnover of dopamine in schizophrenia. Schiz. Bull, 16 (4), 605-615 (1990).
10. Tamminga, C. Schizophrenia and glutamatergic transmission. Critical. Rev. Neurol. 12 (1&2), 21-36 (1998).
11. Angrist, B. & van Kammen D. P. CNS stimulants as a tool in the study of schizophrenia. Trends Neurosci. 7, 388-390 (1984).
12. Swerdlow, N. R. & Koob, G. F. Dopamine, schizophrenia, mania and depression: Toward a unified hypothesms of cortico-striato-pallido-thalamic function. Behav. Brain Sci. 10, 197-245 (1987).
13. Weinberger, D. R. A connectionist approach to the prefrontal cortex. U. of Neuropsychiatry and Clinical Neurosciences 5, 24 1-253 (1993).
14. Bertolino, A et al. Altered development of prefrontal neurons in rhesus monkeys with neonatal mesial temporo-limbic lesions: A proton magnetic resonance spectroscopic imaging study. Cereb. Cortex 7, 740-748 (1997).
15. Jones, G. H., Marsden, C. A. & Robbins, T. W. Increased sensitivity to amphetamine and reward-related stimuli following social isolation in rats: Possible disruption of dopamine-dependent mechanisms of the nucleus accumbens. Psychopharmacology 102, 364-372 (1990).
16. Sams-Dodd, F. Phencyclidine-induced stereotyped behaviour and social isolation in rats: A possible animal model of schizophrenia. Behav. Pharmacol. 7, 3-23 (1996).
17. Snyder, S. H. Psychotogenic drugs as models of schizophrenia. Neuropsychopharm. 1, 197-199 (1988).
18. Wolkin, A. et al. Negative symptoms and hypofrontality in chronic schizophrenia. Arch. Gen. Psychiatry 49, 959-965 (1992).
19. Tarnminga, C. A. et al. Limbic system abnormalities identified in schizophrenia using positron emission tomography with fluorodeoxyglucose and neocortical alterations with deficit syndrome. Arch. Gen. Psychiatry 49 522-530 (1992).
20. Perez, J. L., Shen X., Finkernagel S., Sciorra L., Jenkins, N. A., Gilbert D. J., Copeland, N. G. and Wong, T. W. (1994). Identification and chromosomal mapping of a receptor tyrosine kinase with a putative phospholipid binding sequence in its ectodomain. Oncogene, 9, 211-219.
21. Schramm, M., Falkai, P., Feldmann, N., Knable, M. B. and Bayer, T. A. (1998). Reduced tyrosine kinase receptor C mRNA levels in the frontal cortex of patients with schizophrenia. Neuroscience Letters, 257, 65-68.
22. Bayer, T. A. and Falkai, P. (1997). Schizophrenia a subtle defect in neurotrophic gene function? Neurol, Psychiatr. Brain Res., 5, 151-156.
23. Beasley, C. A. and Reynolds, G. P. (1997). Parvalbuminimmunoreactive neurons are reduced in the prefrontal cortex of schizophrenics. Schiz. Res., 24, 349-355.
24. Danos, P., Baumann, B., Bernstein H-G., Franz, M., Stauch, R., Northoff, G., Krell, D., Falkai, P. and Borgerts, B. (1998). Schizophrenia and anteroventral thalamic nucleus: selective decrease of parvalbuminimmunoreactive thalamocortical projection neurons.
25. Wisen, W & Morris, B. J. In situ hybridisation with synthetic probes. In Insitu hybridisation protocols for the brain. 9-34. Wisden, W & Morris, B. J. (Eds), Academic press, London.

1-63. (canceled)

64. A method for screening a compound which regulates expression of a schizophrenia-related gene(s), said method comprising:
(a) bringing a test compound into contact with transformed cell which enables expression of a gene selected from SEQ ID NO: 1, SEQ ID NO: 2, SEQ ID NO: 3, SEQ ID NO: 4, SEQ ID NO: 5 (PDEIα), SEQ ID NOs: 7, 9 and 11 (CIRL 1, 2 and 3), SEQ ID NO: 13 (trkE), SEQ ID NO: 15 (netrin receptor), SEQ ID NOs: 17 and 19 (synapsin 1A/AB), and SEQ ID NO: 21 (TNFα),
(b) detecting an expression of schizophrenia-relating factor in said cell, and
(c) selecting a compound which promotes or suppresses an induction of the schizophrenia-relating factor in comparison with a control (vehicle).

65. A method for screening a compound which regulates expression of a schizophrenia-related protein(s), said method comprising:
(a) bringing a test compound into contact with transformed cell which enables expression of a schizophrenia-related protein encoded by a nucleic acid selected from SEQ ID NO: 1, SEQ ID NO: 2, SEQ ID NO: 3, SEQ ID NO: 4, SEQ ID NO: 5 (PDEIα), SEQ ID NOs: 7, 9 and 11 (CIRL 1, 2 and 3), SEQ ID NO: 13 (trkE), SEQ ID NO: 15 (netrin receptor), SEQ ID NOs: 17 and 19 (synapsin 1A/AB), and SEQ ID NO: 21 (TNFα),
(b) detecting an expression of schizophrenia-relating protein in said cell, and
(c) selecting a compound which promotes or suppresses expression of the schizophrenia-relating protein in comparison with a control (vehicle).

66. A method of overexpressing a polynucleotide or polypeptide sequence, said method comprising treating a cell with a compound identified by a method according to claim 64 or 65 to produce said overexpression.

67. A method of underexpressing a polynucleotide or polypeptide sequence, said method comprising treating a cell with a compound identified by a method according to claim 64 or 65 to produce said underexpression.

68-76. (canceled)

77. A method of testing a candidate compound for ability to bind one or more polypeptide sequences encodable by one or more polynucleotide sequences selected from SEQ ID NO: 1, SEQ ID NO: 2, SEQ ID NO: 3, SEQ ID NO: 4, SEQ ID NO: 5, SEQ ID NOs: 7, 9 and 11, SEQ ID NO: 13, SEQ ID NO: 15, SEQ ID NOs: 17 and 19, and SEQ ID NO: 21, or fragments thereof, said method comprising contacting the compound with said polypeptide sequence(s) and detecting the presence or absence of a binding interaction.


SEQUENCE LISTING (34 sequences)

SEQ ID NO: 1 - DNA, 291 bp, Rattus sp.
  misc_feature (40)..(40): n is a, c, g, or t
tccagactctgaaagcacacaagaacggtcatggaatctnagcaaagcctaaccaagaaaagctccagttcctcctgttcggcagggcgtgggcatcggcagtgccagggaatgcttggtgcatgaacaggacccccaggtgagccatatttgcagtaagagtcatcagcattgctcctgagaagcctcaggctcagaagaaagcttttgctagcaaattgttagggtctgggaagtaatgctcagggctaggatagcatacccaaggccccgtgctgcatcccaacactg

SEQ ID NO: 2 - DNA, 339 bp, Rattus sp.
aacattccaaacaaaagacactaatatttaggcatgcatgtgatcttgttcaacttctctgtttttagttatttgtgtaaacattatcattacgaattgcattttttgaacttttctattttcaggcaatgagaacaaatacagaggtacagaactaagtattacacacgcacacacacacacacacacacacacacacacagagagagagagagagagagagagagagagagagagagagagagagagagaagggaaggtaaaggtggacttaaaaaacattgctctaaatgggaagtctcaagcaagtctcttcactcagacctcgggggtccttca

SEQ ID NO: 3 - DNA, 343 bp, Rattus sp.
gcaggatgcaaccggacatttctcctttgtagagtgaggatcccacagaagtgttgtgatacccgaagggcagcagcaggctgctgctgtgtctgtgatattaaatcccattgttaatcactaaggattaattgttaaaggataaacacaaggtgtttgtttggctcccagcaatcttgaaattaaataagaaaggaggtttgggaccaactcctgagtgagtagatagccctagaaggaactgcttcacccagaacctgggtcccagggttctagaccagggaggggccaagcaagtgaatggttttgccaggaagctggaactaaggagctgtttggcagg

SEQ ID NO: 4 - DNA, 296 bp, Rattus sp.
cacagcctctgttaaaaggcatctgggtcttggtaaatggctttttatctgtgttatttatgtgtccaacattttatgtgtgtgccgagttcagagggtaagcccacatgctaccacagactcagccagggaaatccagtacaatgggtccaagcacttaattcattaatttatttttgagacagccacgtgtagcccagggtggctttaaactcacaatgtagcagaggctggctttgaactttttatcctcctgattctaattcccatgctagaattagaggcttttgccacca

SEQ ID NO: 5 - DNA, 2658 bp, Rattus sp.
  CDS (1)..(2658)
atggcaagacaaggctgtctcgggtcattccaggtaatatccttgttcacttttgccatcagtgtcaatatctgcttaggattcacagcaagtcgaattaagagggcagaatgggatgaaggacctcccacagtgctgtctgactctccatggaccaacacctctggatcctgcaaaggtagatgctttgagcttcaagaggttggccctccagactgtcggtgtgacaacctgtgtaagagctacagcagctgctgccacgatttcgatgagctctgtttgaaaacagtccgaggctgggagtgcaccaaagacagaagtggggaagtacgaaacgaggaaaatgcctgtcactgcccagaagactgcttgtccaggggagactgctgtaccaactaccaagtggtctgcaaaggagaatcacactgggtagatgatgctgcgagaaatcaaagttccgaatgcctgcaggtttgtccgcctccgttaatcatcttctctgtggatggtttccgtgcatcatacatgaagaaaggcagcaaggttatgcccaacattgagaaactgcggtcctgtggcacccatgtcccctacacgaggcctgtgtaccccacaaaaaccttccctaatctatatacgctggccactggtttatatccggaatcccatggaattgtcggtaattcaatgtatgatcctgtctttgatgcttcgttccatctacgagggcgagagaagtttaatcataggtggtggggaggccaaccgctatggattacagccaccaagcaaggggtgagagctggaacattcttttggtctgtgagcatccctcatgaacggaggatcctaaccattcttcagtggctttctctgccagacaacgagaggccttcagtttatgccttctactcagagcagcctgatttttctggacacaagtacggcccttttggccctgagatgacaaatcctctgagggagattgacaagaccgtggggcagttaatggatggactgaaacaactcaggctgcatcgctgtgtgaacgttatctttgttggagaccatggaatggaagatgtgacatgtgacagaactgagttcttgagcaactatctgactaatgtggatgacattactttagtgcctggaactctgggaagaattcgagccaaatctatcaataattctaaatatgaccctaaaaccattattgctaacctcacgtgcaaaaaaccggatcagcactttaagccttacatgaaacagcaccttcccaaacggttgcactatgccaacaacagaagaattgaagacatccatttattggtcgatcgaagatggcatgttgcaaggaaacctttggacgtttataagaaaccatcaggaaaatgttttttccagggtgaccacggctttgataacaaggtcaatagcatgcagactgttttcgtaggttatggcccaacttttaagtacaggactaaagtgcctccatttgaaaacattgaactttacaatgttatgtgcgatctcctaggcttgaagcccgctcccaataatggaactcatggaagcttgaatcacctactgcgtacaaatacctttaggccaaccatgccagacgaagtcagccgacctaactacccagggattatgtaccttcagtccgagtttgacctgggctgcacctgtgacgataaggtagagccaaagaacaaattggaagaactcaataaacgtcttcataccaaaggatcaacagaagctgaaaccgggaaattcagaggcagcaaacatgaaaacaagaaaaaccttaatggaagtgttgaacctagaaaagagagacatctcctgtatggacggcctgcagtgctctatcggactagctatgatatcttataccatacggactttgaaagtggttatagtgaaatattcttaatgcctctctggacatcgtataccatttctaagcaggctgaggtctccagcatcccagaacacctgaccaactgtgttcgtcctgatgtccgtgtgtctccaggattcagtcagaactgtttagcttataaaaatgataaacagatgtcatatggattcctttttcctccctacctgagctcctccccagaagctaagtatgatgcattcctcgtaaccaacatggttccaatgtaccccgccttcaaacgtgtttgggcttatttccaaagggttttggtgaagaaatatgcttcagaaaggaatggagtcaacgtaataagtggaccgatttttgactacaattacgatggcctacgtgacactgaagatgaaattaaacagtatgtggaaggcagctctatacctgtccccacccactactacagcatcatcaccagctgcctggacttcactcagcctgcagacaagtgtgacggtcccctctctgtgtcttccttcatccttcctcaccgacccgacaatgatgagagctgtaatagctccgaggatgagtcgaagtgggtagaggaactcatgaagatgcacacagctcgggtgcgggacattgagcacctcactggtctggatttctaccggaagactagccgtagctattcggaaattctgaccctcaagacatacctgcatacatatgagagcgagatttaa

SEQ ID NO: 6 - protein, 885 aa, Rattus sp.
MARQGCLGSFQVISLFTFAISVNICLGFTASRIKRAEWDEGPPTVLSDSPWTNTSGSCKGRCFELQEVGPPDCRCDNLCKSYSSCCHDFDELCLKTVRGWECTKDRSGEVRNEENACHCPEDCLSRGDCCTNYQVVCKGESHWVDDAARNQSSECLQVCPPPLIIFSVDGFRASYMKKGSKVMPNIEKLRSCGTHVPYTRPVYPTKTFPNLYTLATGLYPESHGIVGNSMYDPVFDASFHLRGREKFNHRWWGGQPLWITATKQGVRAGTFFWSVSIPHERRILTILQWLSLPDNERPSVYAFYSEQPDFSGHKYGPFGPEMTNPLREIDKTVGQLMDGLKQLRLHRCVNVIFVGDHGMEDVTCDRTEFLSNYLTNVDDITLVPGTLGRIRAKSINNSKYDPKTIIANLTCKKPDQHFKPYMKQHLPKRLHYANNRRIEDIHLLVDRRWHVARKPLDVYKKPSGKCFFQGDHGFDNKVNSMQTVFVGYGPTFKYRTKVPPFENIELYNVMCDLLGLKPAPNNGTHGSLNHLLRTNTFRPTMPDEVSRPNYPGIMYLQSEFDLGCTCDDKVEPKNKLEELNKRLHTKGSTEAETGKFRGSKHENKKNLNGSVEPRKERHLLYGRPAVLYRTSYDILYHTDFESGYSEIFLMPLWTSYTISKQAEVSSIPEHLTNCVRPDVRVSPGFSQNCLAYKNDKQMSYGFLFPPYLSSSPEAKYDAFLVTNMVPMYPAFKRVWAYFQRVLVKKYASERNGVNVISGPIFDYNYDGLRDTEDEIKQYVEGSSIPVPTHYYSIITSCLDFTQPADKCDGPLSVSSFILPHRPDNDESCNSSEDESKWVEELMKMHTARVRDIEHLTGLDFYRKTSRSYSEILTLKTYLHTYESEI

SEQ ID NO: 7 - DNA, 4548 bp, Rattus sp.
  CDS (1)..(4548)
atggcccgcttggctgcagcactctggagtctctgtgtgacgactgtcctcgtcacctctgctacccaaggcctgagccgggctggactcccatttggattgatgcgccgggagctagcatgcgaaggctaccccattgagctgcggtgcccgggcagtgacgtcatcatggtggagaatgcaaactatgggcgcacagatgacaagatctgcgatgccgacccttttcagatggagaacgtgcagtgctacctgcctgacgccttcaaaatcatgtcacagagatgtaataaccgaacccagtgtgtggtggtggccggctctgacgcctttcctgacccctgtcctggaacctacaagtacctggaggtgcagtacgactgtgtcccttacaaagtggagcagaaagtcttcgtgtgcccagggacactgcagaaggtgctggagcccacctccacacatgaatcggagcaccagtctggcgcatggtgcaaggacccactgcaggcaggtgaccgtatctacgttatgccctggatcccctaccgcacggacacactgaccgagtatgcttcctgggaggactatgtggctgcacgccacaccaccacgtacagactgcccaaccgtgtagatggcactggctttgtggtatatgatggtgccgtcttctataacaaggaacgtactcgcaacattgtcaaatatgacctgcggacccgcatcaagagcggagaaacagtcataaacacagccaactaccacgacacctcaccttatcgctggggaggcaaaaccgacattgacctggcagtggatgagaacgggctgtgggtcatctatgccaccgaggggaacaacgggcgtctggtggtgagccagctcaacccctacacactgcgtttcgagggcacctgggaaacaggctatgacaagcgctcagcctccaatgccttcatggtgtgtggtgtcctctatgtgctgcgctctgtttatgtggatgacgacagtgaggcagcaggcaaccgcgtggactatgcctttaacaccaatgcaaaccgagaggagcccgtcagtctcgccttccccaacccctaccagtttgtatcttctgttgactacaatccccgggacaaccagctgtatgtgtggaacaactatttcgtggtgcgctacagcctggagtttggacccccagatcccagtgctggcccagccacttccccacctctcagtaccaccaccacagctcggcctacgcccctcaccagcacagcctcacctgcagccaccactccactccgccgggcgcccctcaccacgcacccagtaggtgccatcaaccagctgggacctgacctgcctccagccacagccccagcacccagtacccggcggcctccagcccccaatctgcatgtgtcccctgagctcttctgtgaaccccgagaggtccggcgggtccagtggccagctacccagcagggtatgctggtagagagaccttgccccaagggaactcgaggaattgcctcgttccagtgcctcccagctctggggctctggaatcctcggggccctgacctcagcaactgcacttccccctgggtcaaccaagtcgcccagaagatcaagagtggagagaatgcagccaacattgctagtgagctggcccgccacacgcggggctccatctatgctggggacgtgtcctcatcggtgaagctgatggagcaactgctagatatcctggatgcccagctccaggccctacggcccattgaacgagagtcagctggcaagaactacaataagatgcacaagcgagagagaacctgcaaggactatatcaaggctgtggtggagacagtggacaacctgcttcggccagaggcacttgagtcatggaaagacatgaatgccaccgaacaggtccatacggccaccatgctcctagatgtcttagaggagggtgccttcctgctggccgacaatgtcagagaacctgctcgcttcttggctgccaagcagaatgtggtcctggaggtcactgtcctgagcacagagggtcaagtgcaggagttggtgttcccccaggagtatgccagtgagagctccattcagctgtccgccaacaccatcaagcagaacagccgcaatggtgtggtgaaggttgtcttcattctctacaacaacctgggcctcttcttgtccacggagaatgccacagtgaagctggcaggtgaggcagggaccggtggccctggaggtgcctccctggtggttaactcacaggtcatcgcagcatccatcaataaggagtccagccgtgtcttcctcatggaccctgtcatctttactgtggcccacttggaggccaagaaccacttcaatgcaaactgctccttctggaactactcagagcgctccatgctgggctactggtcaacccagggctgccgactggtggagtccaataagacccataccacatgtgcctgcagccacctcaccaacttcgcagtgctcatggctcaccgagagatctaccaaggccgtattaatgagctgttgctgtcagtcatcacctgggttggcattgtcatctccctggtctgtctggctatctgcatctccaccttctgcttcctgcggggcctgcagaccgaccgcaacaccatccacaagaacctgtgcatcaacctcttccttgcagagctgctcttcctggttggaatagacaaaactcagtatgaggtcgcctgccctatctttgcgggcctgctgcactacttcttcctggccgccttctcctggctgtgcctagagggcgtgcacctctacctcctgctggtcgaggtgttcgagagcgaatattcacgcaccaagtactattacctgggcggctactgcttcccagccctggtggtaggcatcgcagccgccattgactaccgaagctacggcactgagaaggcctgctggctgagggtggataactatttcatctggagcttcattgggcccgtctcctttgttattgtggtgaacctggtgttcctcatggtgaccctgcacaagatgatccgaagctcatccgtgctcaagcctgactccagccgccttgacaacatcaagtcctgggcgctgggtgccattgcactgctcttcctgctgggcctcacctgggctttcggcctcctcttcatcaacaaggagtcagtagtaatggcttacctcttcacaaccttcaacgccttccagggggtcttcatctttgtctttcactgcgccttacagaaaaaggtgcacaaggagtacagcaagtgcctgcgtcactcctactgctgcattcgctccccacctgggggggctcacggctcccttaagacctcagccatgcgaagtaacacccgctactacacagggacccaggtacccgggcagggaaggcatatccaccaggtctctctggggccgagaggcaggagtgctctgccagagtctcagaaagatcctggagggcagagtggtcctggagaccccctcacgtttgggctgtgtcccagccgaatccggaggatgtggaatgacaccgtgaggaagcagacagagtcgtcctttatggcaggggacatcaacagcacccccaccctgaaccgaggtaccatggggaaccacctactgaccaaccctgtgctacagccccgtgggggcactagcccatacaatacactcattgcagagtctgtgggcttcaatccctcctcgcccccagtcttcaactccccaggaagctacagggaacctaagcaccccttgggcggccgggaagcctgtggcatggacacactgccccttaatggcaacttcaacaacagctactccttgcgaagtggtgatttccctccgggggatgggggtcctgagccaccccgaggccgaaacctagcggatgctgcggcctttgagaagatgatcatctcagagctggtgcacaacaaccttcggggggccagtgggggcgccaaaggtcctccaccagagcctcctgtgccacccgtgccaggagtcagtgaggacgaggctggtgggcctgggggtgctgaccgggctgagattgaacttctctacaaggccctggaggagccactgctgctgccccgggcccagtcggtgctgtaccagagtgatctggatgagtcggagagctgtacggcagaggatggggccaccagccggcccctctcctcccctcccggccgggactccctctatgccagcggggccaacctgcgggactcgccctcctacccggacagcagccccgaagggcctaatgaggccctgccccctcccccacctgctccccctgggcccccagaaatctactacacctctcgcccgccggccctggtggctcggaatcccctacagggctactaccaggtgcggcggcccagccatgagggctacctggcagcccccagccttgaggggccagggcccgatggggatgggcaaatgcagttggtcactagtctctga

SEQ ID NO: 8 - protein, 1515 aa, Rattus sp.
MARLAAALWSLCVTTVLVTSATQGLSRAGLPFGLMRRELACEGYPIELRCPGSDVIMVENANYGRTDDKICDADPFQMENVQCYLPDAFKIMSQRCNNRTQCVVVAGSDAFPDPCPGTYKYLEVQYDCVPYKVEQKVFVCPGTLQKVLEPTSTHESEHQSGAWCKDPLQAGDRIYVMPWIPYRTDTLTEYASWEDYVAARHTTTYRLPNRVDGTGFVVYDGAVFYNKERTRNIVKYDLRTRIKSGETVINTANYHDTSPYRWGGKTDIDLAVDENGLWVIYATEGNNGRLVVSQLNPYTLRFEGTWETGYDKRSASNAFMVCGVLYVLRSVYVDDDSEAAGNRVDYAFNTNANREEPVSLAFPNPYQFVSSVDYNPRDNQLYVWNNYFVVRYSLEFGPPDPSAGPATSPPLSTTTTARPTPLTSTASPAATTPLRRAPLTTHPVGAINQLGPDLPPATAPAPSTRRPPAPNLHVSPELFCEPREVRRVQWPATQQGMLVERPCPKGTRGIASFQCLPALGLWNPRGPDLSNCTSPWVNQVAQKIKSGENAANIASELARHTRGSIYAGDVSSSVKLMEQLLDILDAQLQALRPIERESAGKNYNKMHKRERTCKDYIKAVVETVDNLLRPEALESWKDMNATEQVHTATMLLDVLEEGAFLLADNVREPARFLAAKQNVVLEVTVLSTEGQVQELVFPQEYASESSIQLSANTIKQNSRNGVVKVVFILYNNLGLFLSTENATVKLAGEAGTGGPGGASLVVNSQVIAASINKESSRVFLMDPVIFTVAHLEAKNHFNANCSFWNYSERSMLGYWSTQGCRLVESNKTHTTCACSHLTNFAVLMAHREIYQGRINELLLSVITWVGIVISLVCLAICISTFCFLRGLQTDRNTIHKNLCINLFLAELLFLVGIDKTQYEVACPIFAGLLHYFFLAAFSWLCLEGVHLYLLLVEVFESEYSRTKYYYLGGYCFPALVVGIAAAIDYRSYGTEKACWLRVDNYFIWSFIGPVSFVIVVNLVFLMVTLHKMIRSSSVLKPDSSRLDNIKSWALGAIALLFLLGLTWAFGLLFINKESVVMAYLFTTFNAFQGVFIFVFHCALQKKVHKEYSKCLRHSYCCIRSPPGGAHGSLKTSAMRSNTRYYTGTQVPGQGRHIHQVSLGPRGRSALPESQKDPGGQSGPGDPLTFGLCPSRIRRMWNDTVRKQTESSFMAGDINSTPTLNRGTMGNHLLTNPVLQPRGGTSPYNTLIAESVGFNPSSPPVFNSPGSYREPKHPLGGREACGMDTLPLNGNFNNSYSLRSGDFPPGDGGPEPPRGRNLADAAAFEKMIISELVHNNLRGASGGAKGPPPEPPVPPVPGVSEDEAGGPGGADRAEIELLYKALEEPLLLPRAQSVLYQSDLDESESCTAEDGATSRPLSSPPGRDSLYASGANLRDSPSYPDSSPEGPNEALPPPPPAPPGPPEIYYTSRPPALVARNPLQGYYQVRRPSHEGYLAAPSLEGPGPDGDGQMQLVTSL

SEQ ID NO: 9 - DNA, 4437 bp, Rattus sp.
  CDS (1)..(4437)
atggtgtcttctggttgcagaatgcgaagtctctggtttatcatgataatcagtttctcaccgaataccgaaggtttcagcagagcagccttgccattcgggttagttagacgagagctgtcctgtgaaggttattctatagacctgcgatgtccgggcagtgacgtcatcatgatcgagagcgcaaactacggtcggacggacgacaagatctgcgacgcagacccctttcagatggagaacacagactgctacctccctgatgccttcaaaatcatgactcaaaggtgcaacaaccgaacacagtgtgtagtagttaccgggtcagatgtatttcctgatccatgtcccggaacttacaaataccttgaagttcaatatgaatgtgtcccttacatggagcaaaaagtttttgtgtgtcctggaaccttgaaagcaattgtggactctccaagtatctatgaagctgagcaaaaggcaggtgcttggtgcaaggacccccttcaggctgcagataaaatttattttatgccctggactccctaccgcaccgataccttaatagaatatgcttctttagaagattttcaaaacagccgccagacaacaacatacaaacttccaaaccgagtggacggtactggatttgtggtgtatgacggggcagtcttcttcaacaaagaaagaacgagaaacattgttaaatttgacttgaggactagaatcaagagtggggaggccataatcaactacgccaactaccatgacacctcaccctacagatggggggggaagactgacattgacctggcagtggatgaaaatggcttgtgggtcatctacgccaccgagcagaacaacggaatgatcgtgattagccagctcaatccgtacactctccgattcgaagcaacctgggagacgacgtatgacaagcgtgcggcgtccaatgctttcatgatatgcggggtcctctacgtggtcaggtcagtgtaccaagacaatgaaagcgaagctggcaagaacgtcatcgactacatttacaacacaaggttgagccggggagagcacgtggacgttcccttccccaaccagtaccagtacatcgctgcagtggattacaacccaagagacaaccaactctacgtatggaacaataactttatcttacggtattctctggagtttggtccacccgaccctgcccaagtgcctaccacagctgtgacaataacttcttcagctgagctgttcaaaaccacagtgtcaaccacaagcagtacttcacagagaggccccgtgagcagcacagtcgctggtcctcaggaaggaagccgagggacaaagccacctccagcagtctctacaaccaaaattcctcctgtaacaaatatttttcccctgccagagagattctgcgaagcgttagaaatgaaggggataaagtggcctcagacacaaagggggatgatggttgagcgaccgtgtcccaagggaacaagaggaacggcctcgtatctctgcatggcttccacaggaacctggaacccgaagggcccggatcttagcaactgcacctctcactgggtgaatcagctggcccagaagatcagaagtggagagaatgctgcaagtctggccaacgaactggctaagcacaccaaggggacggtgttcgctggggatgtgagctcctctgtgagactgatggaacagttggtggacatcctggatgcccagctgcaggagctgaaaccgagcgagaaggactcggccgggaggagttataacaagctccaaaaacgagagaagacatgcagggcttaccttaaggccattgtggacacagtagataaccttctgagagccgagactttggactgctggaaacacatgaattcctcagagcaggcgcacacagccaccatgctgttggacaccttggaagaaggagcatttgtcctggcagacaaccttttggaaccaacccgggtctcaatgccaacggataatattgttctagaagtcgctgtcctcagcacggaaggacaggtccaagacttcaccttccatctcggcttcaagggggccttcagctccatccagctctcagccaacaccgtcaagcaaaacagcagaaacgggctggcaaaggtggtattcatcatttaccggagtctgggaccattcctgagcaccgaaaatgcgaccgtcaaactgggcgcagacctcctgggtcggaacagcaccatcgcagtgaactcgcacgtcctttcagtctccatcaataaggagtccagccgtgtgtacttgacagacccggtgcttttttcaatgccacacattgattctgacaattatttcaacgcaaactgctccttctggaactactcagagagaaccatgatgggatattggtctacccagggctgcaagctggttgacactaataaaactcgcacgacgtgtgcatgcagccacctaaccaattttgctattctcatggcccacagggaaattgtgtacaaagatggcgtccacaaattgctgctgacagtcatcacctgggtgggcatcgttgtctccctcgtctgcctggctatctgcatcttcaccttctgcttcttccgaggcctgcaaagcgaccgcaacacgatccacaagaacctgtgtatcaacctcttcatcgctgagtttattttcctaataggcattgataaaacacagtacacgattgcgtgccccgtgtttgcaggactcctgcactttttcttcctggctgctttttcctggatgtgcctagaaggtgtgcagctctacctcatgttggttgaagttttcgagagtgaatactcaaggaagaagtattactatgtcgccgggtacctcttccctgccacagtggtcggtgtttcagctgctatcgactacaagagttacgggacactagaggcttgctggcttcacgttgataactatttcatatggagtttcattgggcctgttactttcatcattctgctaaatattattttcctggtgatcacgctgtgcaaaatggtgaaacattcaaacactttgaaaccagattctagcaggttggaaaacattaataattaccgtgtttgtgatggatactataatacggacttacctgggtcttgggtgctcggtgcgttcgccctgctgtgtctcctgggcctaacctggtcctttgggttgctttttgttaacgaggagaccgttgtcatggcttatctcttcaccgcctttaatgctttccagggactgtttattttcatcttccactgtgctcttcaaaagaaagtacggaaagagtatgccaagtgcttcagacactggtactgctgtggtggcctcccgaccgagagcccgcacagctctgtaaaggcgtccacctcccgcaccagtgctcgttactcctctggtacacagagccgtataagaaggatgtggaatgacaccgtgaggaagcagtctgaatcgtcttttatctcaggtgacatcaatagcacttctacccttaatcaaggaatgactggcaattacctactaacaaaccctcttcttcgaccccacggcactaacaacccctataacacattgctcgctgaaacagttgtatgtaatgccccttcagcgcccgtgtttaactcaccaggacattcactgaacaatacccgggacaccagcgccatggatactctaccgctaaatggtaacttcaacaacagctactccctgcgcaaggccgactaccacgacggcgtgcaggttgtggactgtggactaagtctgaacgacaccgcgtttgagaaaatgatcatttcagagttagtgcacaacaacctccggggtagcaacaaaacccacaacttggagctcaagctcccagttaaacccgtgattggcggcagcagcagcgaagatgacgcgatcgtggccgacgcctcatctttgatgcacggtgataacccagggctggaattccgccacaaagagctggaggcaccgctcatccctcagcggactcactcgcttctgtaccaaccccagaaaaaagtgaaacccgaggcaaccgacagctacgtctcccagctgacggccgaggccgacgagcacctccagtcccccaacagagactctctgtacacgagcatgcccaacctaagagactctccctacccggagagcagcccggacatggcagaggacctgtctccctccaggaggagcgagaacgaggacatttactacaaaagtatgcccaatcttggggctggccgccagctccagatgtgctaccagatcagcagaggcaatagcgatggctacatcatccccattaacaaagaagggtgcatcccagagggggacgtcagggaaggacagatgcagctggtaacaagtctttaa

SEQ ID NO: 10 - protein, 1478 aa, Rattus sp.
MVSSGCRMRSLWFIMIISFSPNTEGFSRAALPFGLVRRELSCEGYSIDLRCPGSDVIMIESANYGRTDDKICDADPFQMENTDCYLPDAFKIMTQRCNNRTQCVVVTGSDVFPDPCPGTYKYLEVQYECVPYMEQKVFVCPGTLKAIVDSPSIYEAEQKAGAWCKDPLQAADKIYFMPWTPYRTDTLIEYASLEDFQNSRQTTTYKLPNRVDGTGFVVYDGAVFFNKERTRNIVKFDLRTRIKSGEAIINYANYHDTSPYRWGGKTDIDLAVDENGLWVIYATEQNNGMIVISQLNPYTLRFEATWETTYDKRAASNAFMICGVLYVVRSVYQDNESEAGKNVIDYIYNTRLSRGEHVDVPFPNQYQYIAAVDYNPRDNQLYVWNNNFILRYSLEFGPPDPAQVPTTAVTITSSAELFKTTVSTTSSTSQRGPVSSTVAGPQEGSRGTKPPPAVSTTKIPPVTNIFPLPERFCEALEMKGIKWPQTQRGMMVERPCPKGTRGTASYLCMASTGTWNPKGPDLSNCTSHWVNQLAQKIRSGENAASLANELAKHTKGTVFAGDVSSSVRLMEQLVDILDAQLQELKPSEKDSAGRSYNKLQKREKTCRAYLKAIVDTVDNLLRAETLDCWKHMNSSEQAHTATMLLDTLEEGAFVLADNLLEPTRVSMPTDNIVLEVAVLSTEGQVQDFTFHLGFKGAFSSIQLSANTVKQNSRNGLAKVVFIIYRSLGPFLSTENATVKLGADLLGRNSTIAVNSHVLSVSINKESSRVYLTDPVLFSMPHIDSDNYFNANCSFWNYSERTMMGYWSTQGCKLVDTNKTRTTCACSHLTNFAILMAHREIVYKDGVHKLLLTVITWVGIVVSLVCLAICIFTFCFFRGLQSDRNTIHKNLCINLFIAEFIFLIGIDKTQYTIACPVFAGLLHFFFLAAFSWMCLEGVQLYLMLVEVFESEYSRKKYYYVAGYLFPATVVGVSAAIDYKSYGTLEACWLHVDNYFIWSFIGPVTFIILLNIIFLVITLCKMVKHSNTLKPDSSRLENINNYRVCDGYYNTDLPGSWVLGAFALLCLLGLTWSFGLLFVNEETVVMAYLFTAFNAFQGLFIFIFHCALQKKVRKEYAKCFRHWYCCGGLPTESPHSSVKASTSRTSARYSSGTQSRIRRMWNDTVRKQSESSFISGDINSTSTLNQGMTGNYLLTNPLLRPHGTNNPYNTLLAETVVCNAPSAPVFNSPGHSLNNTRDTSAMDTLPLNGNFNNSYSLRKADYHDGVQVVDCGLSLNDTAFEKMIISELVHNNLRGSNKTHNLELKLPVKPVIGGSSSEDDAIVADASSLMHGDNPGLEFRHKELEAPLIPQRTHSLLYQPQKKVKPEATDSYVSQLTAEADEHLQSPNRDSLYTSMPNLRDSPYPESSPDMAEDLSPSRRSENEDIYYKSMPNLGAGRQLQMCYQISRGNSDGYIIPINKEGCIPEGDVREGQMQLVTSL

SEQ ID NO: 11 - DNA, 4584 bp, Rattus sp.
  CDS (1)..(4584)
atgtgtccacctcagctgttcatcctcatgatgcttttagcacctgtagttcatggtggcaagcacaatgagagacatccagccctcgctgctccactgcgacatgctgagcacagcccaggaggccctctccctcccagacatcttcttcagcagccagctgcagagcgctctacagctcatcgaggacaagggccacgtggaactgccagaggagttcgcggacccggtgccccaggagcacagattgcagcccaagctttcagccgtgccccaattcccatggcagtggtccgcagagagctctcctgtgagagctaccccattgagctacgctgtccaggcacagacgtcatcatgatcgaaagcgccaactacgggaggacagatgacaagatctgtgactcggaccctgctcagatggagaatattcggtgttatctgccagatgcctataagattatgtctcaaagatgcaataacagaacccagtgtgcagtggtggcaggtcctgatgtatttccagacccatgtccgggaacatataaataccttgaagtgcagtatgaatgtgtcccttacaaagtggaacaaaaagtttttctttgtcccggactgctcaaaggagtgtaccagagcgaacacttgtttgaatctgaccaccaatctggggcatggtgcaaagaccctctacaggcttctgacaagatttactatatgccctggactccctacagaaccgataccctgacagagtattcatccaaagatgacttcattgctggaaggccgacaactacatacaagctccctcacagagtggatggtactggatttgtagtatatgatggtgccctgttcttcaacaaggagcgtacaaggaacatagtaaagtttgatttgaggactaggataaagagtggagaggcaatcatagcaaatgctaactaccatgatacctccccataccgatggggtggcaagtccgacatagacttggcagtggatgaaaacggattatgggtaatctatgcaacagaacagaacaatggcaaaattgttattagccagttgaacccttacaccctacggattgaggggacatgggacactgcctatgataaaaggtctgcttccaatgcatttatgatttgtgggattctgtatgtggtcaagtctgtatatgaggatgacgacaatgaggccaccggtaataagattgactacatttacaatactgaccaaagcaaggatagcctggtggatgtaccctttcccaactcataccagtacatagcagccgtggactacaatcccagggacaatctgctgtacgtgtggaacaactaccatgttgtcaaatactccttggacttcgggcctctggatagcagatcagggccagtgcatcatggacaagtttcctacatctctccaccgattcaccttgactctgacctggaaaggccccctgtcagagggatttctaccacaggacccctgggtatgggaagcacgaccaccagcaccaccctccggactaccacctggaacctagggaggagtacaacgccatccttgcctggcagaagaaaccgcagtaccagtacgccgtccccagcgattgaggtgctggatgttaccacacacctgccatctgcagcctcccaaatcccagcgatggaagagagctgcgaggctgtggaagcccgagagatcatgtggtttaagacccgacaggggcaagtagcaaagcagtcatgcccagcaggaaccataggtgtatcaacttacctgtgtcttgctcctgatggaatatgggatccccaaggaccagatctcagcaactgctcttctccttgggtcaatcacataacacagaagctgaaatctggagaaacagctgccaatattgccagagagctagcagaacagaccagaaatcatttgaacgccggggatatcacctactcagttcgtgccatggaccaactggttggcctcctggacgtacagctcaggaatttgacaccaggggggaaggacagtgctgcccgaagcttgaacaagcttcagaaaagagagcgctcttgcagagcctatgtccaggcgatggtggagacagttaacaatctccttcagccacaagctctgaatgcgtggagggacctgacgacaagtgatcaactacgcgcagccaccatgttgctcgacactgtggaggagagtgctttcgtgttagccgataaccttttgaagaccgacattgtcagggagaatacagacaatattcagttggaggttgcaaggctgagcacggaaggaaacctagaagatctaaaatttccagaaaacacgggccacggaagcactatacagctttccgcaaacacgttaaagcaaaatggccggaatggagagattagagtggcctttgtcctgtataacaacctgggtccttatttatctacggagaatgccagtatgaagttgggcacagaagctatgtccacaaatcactcagttatcgtcaattcccctgttattacagcagcaataaataaggaattcagtaataaagtgtatttggctgatcctgtggtatttactgttaaacatatcaagcagtcagaggaaaatttcaaccctaactgttcattttggagctattccaagcgcacaatgacaggttattggtcaacacaaggctgtcgactcctgacaacgaacaagacacacactacgtgctcctgtaaccacctcaccaacttcgcagtattaatggcacatgtggaagttaagcacagcgatgccgtccacgatcttcttctggatgtgatcacgtgggtcggaatcctgttgtctcttgtttgtctcctgatctgcatcttcacattctgcttcttccgtgggctccagagcgaccgtaacaccattcacaagaacctgtgcatcagcctgtttgtggcagaactgctcttcctgattgggatcaacagaaccgaccaaccgattgcctgtgcagtgtttgcggctcttttgcatttcttcttcttggcggccttcacctggatgtttctagaaggggtacagctgtatatcatgctggtggaggtctttgagagtgagcattcccgtaggaagtacttctatctggttggctacgggatgcccgcactcatcgtggccgtttctgctgcagtcgactacaggagctatggaacagacaaagtatgttggcttcgccttgacacctacttcatttggagttttataggaccagcgaccttgataattatgctgaatgtaatcttcctcgggattgctttatacaaaatgtttcaccatactgccatactgaaacctgaatcaggctgtcttgataatatcaagtcatgggttataggtgcaatagcgctgctctgcctattaggattgacctgggcctttggactcatgtatattaatgaaagcacagtcatcatggcgtatctcttcaccattttcaattctctacaggggatgtttatattcattttccactgtgtcctacagaagaaggtacggaaagagtatgggaaatgcctacggacgcattgctgtagtgggaaaagcacggagagttcgattggctcagggaaaacatctggttctcgaactccaggacggtattccacaggctcacagagccggattcggagaatgtggaatgacaccgtccgaaagcagtcagagtcatccttcatcactggagacataaacagctcagcgtcgctcaacagagaggggcttctgaacaatgccagggatacaagtgtcatggatactctaccactgaatggtaaccatggcaacagttacagcattgctggcggcgaatacctgagcaactgtgtgcaaattatagaccgtggctataaccacaacgagaccgccctagaaaaaaagatcctaaaggaactcacttccaactatatcccttcatacctgaacaaccacgagcgctccagcgaacagaaccggaacatgatgaacaaactggtggacaacttaggcagtgggagtgaagatgacgccatagtcctggatgacgcagcgtcctttaaccacgaggagagtctgggcctggaactcattcacgaggaatcggatgctcccttgctgcccccgagggtttactccaccgataaccaccagccacaccattacagcaggaggcggctcccccaggaccacagcgagagcttcttccctctgctaaccgacgagcacacagaagacccgcagtcaccgcacagggactctctgtacaccagcatgccggccctggccggtgtgcccgctgcagacagtgtgaccaccagcacccagaccgaagccgcagcggccaagggtggtgacgccgaagatgtttactacaaaagcatgccaaacctgggctccagaaaccatgtgcacccgctgcacgcctactaccagctagggcgaggcagcagcgatggattcatagttcctcccaataaagatggggcctctccggaggggacttccaaaggaccggcgcacttggtcactagtctatag

SEQ ID NO: 12 - protein, 1527 aa, Rattus sp.
MCPPQLFILMMLLAPVVHGGKHNERHPALAAPLRHAEHSPGGPLPPRHLLQQPAAERSTAHRGQGPRGTARGVRGPGAPGAQIAAQAFSRAPIPMAVVRRELSCESYPIELRCPGTDVIMIESANYGRTDDKICDSDPAQMENIRCYLPDAYKIMSQRCNNRTQCAVVAGPDVFPDPCPGTYKYLEVQYECVPYKVEQKVFLCPGLLKGVYQSEHLFESDHQSGAWCKDPLQASDKIYYMPWTPYRTDTLTEYSSKDDFIAGRPTTTYKLPHRVDGTGFVVYDGALFFNKERTRNIVKFDLRTRIKSGEAIIANANYHDTSPYRWGGKSDIDLAVDENGLWVIYATEQNNGKIVISQLNPYTLRIEGTWDTAYDKRSASNAFMICGILYVVKSVYEDDDNEATGNKIDYIYNTDQSKDSLVDVPFPNSYQYIAAVDYNPRDNLLYVWNNYHVVKYSLDFGPLDSRSGPVHHGQVSYISPPIHLDSDLERPPVRGISTTGPLGMGSTTTSTTLRTTTWNLGRSTTPSLPGRRNRSTSTPSPAIEVLDVTTHLPSAASQIPAMEESCEAVEAREIMWFKTRQGQVAKQSCPAGTIGVSTYLCLAPDGIWDPQGPDLSNCSSPWVNHITQKLKSGETAANIARELAEQTRNHLNAGDITYSVRAMDQLVGLLDVQLRNLTPGGKDSAARSLNKLQKRERSCRAYVQAMVETVNNLLQPQALNAWRDLTTSDQLRAATMLLDTVEESAFVLADNLLKTDIVRENTDNIQLEVARLSTEGNLEDLKFPENTGHGSTIQLSANTLKQNGRNGEIRVAFVLYNNLGPYLSTENASMKLGTEAMSTNHSVIVNSPVITAAINKEFSNKVYLADPVVFTVKHIKQSEENFNPNCSFWSYSKRTMTGYWSTQGCRLLTTNKTHTTCSCNHLTNFAVLMAHVEVKHSDAVHDLLLDVITWVGILLSLVCLLICIFTFCFFRGLQSDRNTIHKNLCISLFVAELLFLIGINRTDQPIACAVFAALLHFFFLAAFTWMFLEGVQLYIMLVEVFESEHSRRKYFYLVGYGMPALIVAVSAAVDYRSYGTDKVCWLRLDTYFIWSFIGPATLIIMLNVIFLGIALYKMFHHTAILKPESGCLDNIKSWVIGAIALLCLLGLTWAFGLMYINESTVIMAYLFTIFNSLQGMFIFIFHCVLQKKVRKEYGKCLRTHCCSGKSTESSIGSGKTSGSRTPGRYSTGSQSRIRRMWNDTVRKQSESSFITGDINSSASLNREGLLNNARDTSVMDTLPLNGNHGNSYSIAGGEYLSNCVQIIDRGYNHNETALEKKILKELTSNYIPSYLNNHERSSEQNRNMMNKLVDNLGSGSEDDAIVLDDAASFNHEESLGLELIHEESDAPLLPPRVYSTDNHQPHHYSRRRLPQDHSESFFPLLTDEHTEDPQSPHRDSLYTSMPALAGVPAADSVTTSTQTEAAAAKGGDAEDVYYKSMPNLGSRNHVHPLHAYYQLGRGSSDGFIVPPNKDGASPEGTSKGPAHLVTSL

SEQ ID NO: 13 - DNA, 3554 bp, Homo sapiens
  CDS (46)..(2676)
gagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactccacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagtgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggtcagtcttgatttcccccttaatgtgcgtaagggacaccctttgctggtagctgtcaagatcttacggccagatgccaccaagaatgccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctagccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggcagctcaccgacgagcaggtcatcgagaacgcgggggagttcttccgggaccagggccggcaggtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagtgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacctgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaaaaaaaa

SEQ ID NO: 14 - protein, 876 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 15 - DNA, 2697 bp, Rattus sp.
  CDS (1)..(2697)
atggccgtccggcccggcctgtggccagtgctcctgggcatagtcctcgccgcctggcttcgtggttcgggtgcccagcagagtgccacggtggccaatccagtgcccggtgccaaccccgacctgctgccccacttcctggtagagcctgaggacgtgtacattgtcaagaacaagccggtgttgttggtgtgcaaggctgtgcctgccacccagatcttcttcaagtgcaatggggaatgggtccgccaggtcgatcacgtaattgaacgcagcaccgacagcagcagcggattgccaaccatggaggtccgtatcaacgtatcgaggcagcaggtagagaaagtgtttgggctggaggaatactggtgccagtgtgtggcatggagctcctcgggtaccaccaaaagtcagaaggcctacatccggattgcctatttgcgcaagaactttgagcaggagccactggccaaggaagtgtcactggagcaaggcattgtactaccttgtcgccccccagaaggaatccccccagctgaggtggagtggcttcgaaatgaggacctcgtggacccctccctcgatcccaatgtgtacatcacgcgggagcacagcctagtcgtgcgtcaggcccgcctggccgacacggccaactacacctgtgtggccaagaacatcgtagcccgtcgccgaagcacctctgcagcggtcattgtttatgtgaacggtgggtggtcgacgtggactgagtggtccgtctgcagcgccagctgtgggcgtggctggcagaaacggagccggagctgcaccaacccggcacctctcaacgggggcgccttctgtgaggggcagaatgtccagaaaacagcctgcgccactctgtgcccagtggatgggagctggagttcgtggagtaagtggtcagcctgtgggcttgactgcacccactggcggagccgcgagtgctctgacccagcaccccgcaatggaggtgaggagtgtcggggtgctgacctggacacccgcaactgtaccagtgacctctgcctgcacaccgcttcttgccccgaggacgtggctctctacatcggccttgtcgctgtggctgtgtgcctcttcttgctgttgctggcccttggactcatttactgtcgcaagaaggaagggctggactccgatgtggccgactcgtccatcctcacctcgggcttccagcctgtcagcatcaagcccagcaaagcagacaacccccacctgctcaccatccagccagacctcagcaccaccactaccacctaccagggcagtctatgttcgaggcaggatggacccagccccaagttccagctctctaatggtcacctgctcagcccactggggagtggccgccatacgttgcaccacagctcacccacctctgaggctgaggacttcgtctcccgcctctccacccaaaactactttcgttccctgccccgcggcaccagcaacatggcctacgggaccttcaacttcctcgggggccggctgatgatccctaatacggggatcagcctcctcatacccccggatgccatcccccgaggaaagatctacgagatctacctcacactgcacaagccagaagacgtgaggttgcccctagctggctgtcagaccctgctgagtccagtcgttagctgtgggcccccaggagtcctgctcacccggccagtcatccttgcaatggaccactgtggagagcccagccctgacagctggagtctgcgcctcaaaaagcagtcctgcgagggcagttgggaggatgtgctgcaccttggtgaggagtcaccttcccacctctactactgccagctggaggccggggcctgctatgtcttcacggagcagctgggccgctttgccctggtaggagaggccctcagcgtggctgccaccaagcgcctcaggctccttctgtttgctcccgtggcctgtacgtcccttgagtacaacatccgagtgtactgcctacacgacacccacgacgctctcaaggaggtggtgcagctggagaagcagctaggtggacagctgatccaggagcctcgcgtcctgcacttcaaagacagttaccacaacctacgtctctccatccacgacgtgcccagctccctgtggaagagcaagctacttgtcagctaccaggagatccctttttaccacatctggaacggcacccagcagtatctgcactgcaccttcaccctggagcgcatcaacgccagcaccagcgacctggcctgcaaggtgtgggtgtggcaggtggagggagatgggcagagcttcaacatcaacttcaacatcactaaggacacaaggtttgctgaattgttggctctggagagtgaagggggggtcccagccctggtgggccccagtgccttcaagatccccttcctcattcggcaaaagatcatcgccagtctggacccaccctgcagccggggcgccgactggagaactctagcccagaaacttcacctggacagccatcttagcttctttgcctccaagcccagccctacagccatgatcctcaacctatgggaggcacggcacttccccaacggcaacctcggccagctggcagcagctgtggccggactgggccaaccagatgctggcctcttcacggtgtcggaggccgagtgttga

SEQ ID NO: 16 - protein, 898 aa, Rattus sp.
MAVRPGLWPVLLGIVLAAWLRGSGAQQSATVANPVPGANPDLLPHFLVEPEDVYIVKNKPVLLVCKAVPATQIFFKCNGEWVRQVDHVIERSTDSSSGLPTMEVRINVSRQQVEKVFGLEEYWCQCVAWSSSGTTKSQKAYIRIAYLRKNFEQEPLAKEVSLEQGIVLPCRPPEGIPPAEVEWLRNEDLVDPSLDPNVYITREHSLVVRQARLADTANYTCVAKNIVARRRSTSAAVIVYVNGGWSTWTEWSVCSASCGRGWQKRSRSCTNPAPLNGGAFCEGQNVQKTACATLCPVDGSWSSWSKWSACGLDCTHWRSRECSDPAPRNGGEECRGADLDTRNCTSDLCLHTASCPEDVALYIGLVAVAVCLFLLLLALGLIYCRKKEGLDSDVADSSILTSGFQPVSIKPSKADNPHLLTIQPDLSTTTTTYQGSLCSRQDGPSPKFQLSNGHLLSPLGSGRHTLHHSSPTSEAEDFVSRLSTQNYFRSLPRGTSNMAYGTFNFLGGRLMIPNTGISLLIPPDAIPRGKIYEIYLTLHKPEDVRLPLAGCQTLLSPVVSCGPPGVLLTRPVILAMDHCGEPSPDSWSLRLKKQSCEGSWEDVLHLGEESPSHLYYCQLEAGACYVFTEQLGRFALVGEALSVAATKRLRLLLFAPVACTSLEYNIRVYCLHDTHDALKEVVQLEKQLGGQLIQEPRVLHFKDSYHNLRLSIHDVPSSLWKSKLLVSYQEIPFYHIWNGTQQYLHCTFTLERINASTSDLACKVWVWQVEGDGQSFNINFNITKDTRFAELLALESEGGVPALVGPSAFKIPFLIRQKIIASLDPPCSRGADWRTLAQKLHLDSHLSFFASKPSPTAMILNLWEARHFPNGNLGQLAAAVAGLGQPDAGLFTVSEAEC

SEQ ID NO: 17 - DNA, 2115 bp, Rattus sp.
  CDS (1)..(2115)
atgaactacctgcggcgccgcctgtcggacagcaacttcatggccaatctgcctaatgggtatatgacagacctgcagcgcccgcaaccacctccgccgccgccctcagccgccagcccaggggccactcccggatccgctgctgcctctgctgagagggcctccacagctgctccagtggcctctccagcagcccctagtcccgggtcctcggggggcggtggcttcttctcctcgctgtctaacgcggtcaaacagaccacagcagctgcagccgccaccttcagtgagcaggtgggcggtggctctgggggcgcaggccgcgggggcgccgccgccagggtgctgctggtcatcgacgagccgcacaccgactgggcaaaatacttcaaagggaagaagatccatggagaaattgacattaaagtagagcaagctgaattctccgatctcaatcttgtggctcatgccaatggtggattctccgtggacatggaagttcttcggaatggggtcaaagttgtgaggtctctgaagccagactttgtgctgatccgccagcatgccttcagcatggcacgtaatggagactaccgcagtttggtcattgggctgcagtatgctgggatccccagtgttaactctttgcattctgtctacaacttttgtgacaaaccctgggtgtttgcccagatggttcgactacacaagaagcttggaacagaggaattccctctgattgatcagactttctatcccaatcataaagagatgctcagcagcacaacataccctgtagttgtgaagatgggccacgcacactctgggatgggcaaggtcaaggtagacaaccaacatgacttccaggatattgcaagtgttgtggcactgactaagacatatgccactgctgagcccttcattgatgctaaatacgatgtgcgtgtccagaagattgggcagaactacaaggcctacatgaggacatcagtgtcagggaactggaagaccaatacaggctctgctatgcttgagcagattgctatgtctgacaggtacaagttgtgggtagacacgtgctcagagatttttgggggacttgacatctgcgcagtggaagcactgcatggcaaggacggaagggatcacattattgaggtggtgggctcctccatgccactcattggggatcaccaggatgaagacaagcagctcatcgtggaacttgtggtcaacaagatgactcaggctctgcctcggcagcgggatgcttcccctggcaggggttcccacagccagactccatccccaggagccctgcccttgggccgccagacctcccagcagcctgcaggacctcctgctcaacaacgacccccaccccagggaggccctccacaaccaggcccaggacctcagcgccagggacccccgctgcaacagcgcccacccccacaaggccagcaacatctttctggccttggacccccagctggcagccctctgccccagcgcctaccaagtcccacagcagcacctcagcagtccgcctctcaggccacaccaatgacccagggtcaaggccgccagtcacggccagtggcaggaggccccggagcacctccagcagcccgcccgccggcctccccatctccacagcgtcaggcagggcccccacaggctacccgtcaggcatctatctctggtccagctccaccgaaggtctcaggagcctcacccggagggcagcagcgccaaggccctccccagaaacccccaggccctgctggtcccattcgtcaggccagccaggcaggtcccggacctcgcactgggccacccaccacacagcagccccggcccagcggcccaggtcctgctggacgtcccaccaaaccacagctggctcagaaacccagccaggatgtgccaccacccatcattgctgctgccgggggacccccgcacccccagctcaacaaatcccagtctctgaccaatgccttcaaccttccagagccagccccgcccaggcccagccttagccaggatgaggtgaaagctgagaccatccgcagcctgaggaagtctttcgccagcctcttctccgactga

SEQ ID NO: 18 - protein, 704 aa, Rattus sp.
MNYLRRRLSDSNFMANLPNGYMTDLQRPQPPPPPPSAASPGATPGSAAASAERASTAAPVASPAAPSPGSSGGGGFFSSLSNAVKQTTAAAAATFSEQVGGGSGGAGRGGAAARVLLVIDEPHTDWAKYFKGKKIHGEIDIKVEQAEFSDLNLVAHANGGFSVDMEVLRNGVKVVRSLKPDFVLIRQHAFSMARNGDYRSLVIGLQYAGIPSVNSLHSVYNFCDKPWVFAQMVRLHKKLGTEEFPLIDQTFYPNHKEMLSSTTYPVVVKMGHAHSGMGKVKVDNQHDFQDIASVVALTKTYATAEPFIDAKYDVRVQKIGQNYKAYMRTSVSGNWKTNTGSAMLEQIAMSDRYKLWVDTCSEIFGGLDICAVEALHGKDGRDHIIEVVGSSMPLIGDHQDEDKQLIVELVVNKMTQALPRQRDASPGRGSHSQTPSPGALPLGRQTSQQPAGPPAQQRPPPQGGPPQPGPGPQRQGPPLQQRPPPQGQQHLSGLGPPAGSPLPQRLPSPTAAPQQSASQATPMTQGQGRQSRPVAGGPGAPPAARPPASPSPQRQAGPPQATRQASISGPAPPKVSGASPGGQQRQGPPQKPPGPAGPIRQASQAGPGPRTGPPTTQQPRPSGPGPAGRPTKPQLAQKPSQDVPPPIIAAAGGPPHPQLNKSQSLTNAFNLPEPAPPRPSLSQDEVKAETIRSLRKSFASLFSD

SEQ ID NO: 19 - DNA, 2007 bp, Rattus sp.
  CDS (1)..(2007)
atgaactacctgcggcgccgcctgtcggacagcaacttcatggccaatctgcctaatgggtatatgacagacctgcagcgcccgcaaccacctccgccgccgccctcagccgccagcccaggggccactcccggatccgctgctgcctctgctgagagggcctccacagctgctccagtggcctctccagcagcccctagtcccgggtcctcggggggcggtggcttcttctcctcgctgtctaacgcggtcaaacagaccacagcagctgcagccgccaccttcagtgagcaggtgggcggtggctctgggggcgcaggccgcgggggcgccgccgccagggtgctgctggtcatcgacgagccgcacaccgactgggcaaaatacttcaaagggaagaagatccatggagaaattgacattaaagtagagcaagctgaattctccgatctcaatcttgtggctcatgccaatggtggattctccgtggacatggaagttcttcggaatggggtcaaagttgtgaggtctctgaagccagactttgtgctgatccgccagcatgccttcagcatggcacgtaatggagactaccgcagtttggtcattgggctgcagtatgctgggatccccagtgttaactctttgcattctgtctacaacttttgtgacaaaccctgggtgtttgcccagatggttcgactacacaagaagcttggaacagaggaattccctctgattgatcagactttctatcccaatcataaagagatgctcagcagcacaacataccctgtagttgtgaagatgggccacgcacactctgggatgggcaaggtcaaggtagacaaccaacatgacttccaggatattgcaagtgttgtggcactgactaagacatatgccactgctgagcccttcattgatgctaaatacgatgtgcgtgtccagaagattgggcagaactacaaggcctacatgaggacatcagtgtcagggaactggaagaccaatacaggctctgctatgcttgagcagattgctatgtctgacaggtacaagttgtgggtagacacgtgctcagagatttttgggggacttgacatctgcgcagtggaagcactgcatggcaaggacggaagggatcacattattgaggtggtgggctcctccatgccactcattggggatcaccaggatgaagacaagcagctcatcgtggaacttgtggtcaacaagatgactcaggctctgcctcggcagcgggatgcttcccctggcaggggttcccacagccagactccatccccaggagccctgcccttgggccgccagacctcccagcagcctgcaggacctcctgctcaacaacgacccccaccccagggaggccctccacaaccaggcccaggacctcagcgccagggacccccgctgcaacagcgcccacccccacaaggccagcaacatctttctggccttggacccccagctggcagccctctgccccagcgcctaccaagtcccacagcagcacctcagcagtccgcctctcaggccacaccaatgacccagggtcaaggccgccagtcacggccagtggcaggaggccccggagcacctccagcagcccgcccgccggcctccccatctccacagcgtcaggcagggcccccacaggctacccgtcaggcatctatctctggtccagctccaccgaaggtctcaggagcctcacccggagggcagcagcgccaaggccctccccagaaacccccaggccctgctggtcccattcgtcaggccagccaggcaggtcccggacctcgcactgggccacccaccacacagcagccccggcccagcggcccaggtcctgctggacgtcccaccaaaccacagctggctcagaaacccagccaggatgtgccaccacccatcattgctgctgccgggggacccccgcacccccagctcaaagccagccccgcccaggcccagccttag

SEQ ID NO: 20 - protein, 668 aa, Rattus sp.
MNYLRRRLSDSNFMANLPNGYMTDLQRPQPPPPPPSAASPGATPGSAAASAERASTAAPVASPAAPSPGSSGGGGFFSSLSNAVKQTTAAAAATFSEQVGGGSGGAGRGGAAARVLLVIDEPHTDWAKYFKGKKIHGEIDIKVEQAEFSDLNLVAHANGGFSVDMEVLRNGVKVVRSLKPDFVLIRQHAFSMARNGDYRSLVIGLQYAGIPSVNSLHSVYNFCDKPWVFAQMVRLHKKLGTEEFPLIDQTFYPNHKEMLSSTTYPVVVKMGHAHSGMGKVKVDNQHDFQDIASVVALTKTYATAEPFIDAKYDVRVQKIGQNYKAYMRTSVSGNWKTNTGSAMLEQIAMSDRYKLWVDTCSEIFGGLDICAVEALHGKDGRDHIIEVVGSSMPLIGDHQDEDKQLIVELVVNKMTQALPRQRDASPGRGSHSQTPSPGALPLGRQTSQQPAGPPAQQRPPPQGGPPQPGPGPQRQGPPLQQRPPPQGQQHLSGLGPPAGSPLPQRLPSPTAAPQQSASQATPMTQGQGRQSRPVAGGPGAPPAARPPASPSPQRQAGPPQATRQASISGPAPPKVSGASPGGQQRQGPPQKPPGPAGPIRQASQAGPGPRTGPPTTQQPRPSGPGPAGRPTKPQLAQKPSQDVPPPIIAAAGGPPHPQLKASPAQAQP

SEQ ID NO: 21 - DNA, 705 bp, Rattus sp.
  CDS (1)..(705)
atgagcacagaaagcatgatccgagatgtggaactggcagaggaggcgctccccaaaaagatggggggcctccagaactccaggcggtgtctgtgcctcagcctcttctcattcctgctcgtggcgggggccaccacgctcttctgtctactgaacttcggggtgatcggtcccaacaaggaggagaagttcccaaatgggctccctctcatcagttccatggcccagaccctcacactcagatcatcttctcaaaactcgagtgacaagcccgtagcccacgtcgtagcaaaccaccaagcagaggagcagctggagtggctgagccagcgtgccaacgccctcctggccaatggcatggatctcaaagacaaccaactggtggtaccagcagatgggctgtaccttatctactcccaggttctcttcaagggacaaggctgccccgactatgtgctcctcacccacaccgtcagccgatttgccatttcataccaggagaaagtcagcctcctctccgccatcaagagcccttgccctaaggacacccctgagggagctgagctcgagccctggtatgagcccatgtacctgggaggagtcttccagctggagaagggggacctgctcagcgctgaggtcaacctgcccaagtacttagacatcacggagtccgggcaggtctactttggagtcattgctctg

SEQ ID NO: 22 - protein, 235 aa, Rattus sp.
MSTESMIRDVELAEEALPKKMGGLQNSRRCLCLSLFSFLLVAGATTLFCLLNFGVIGPNKEEKFPNGLPLISSMAQTLTLRSSSQNSSDKPVAHVVANHQAEEQLEWLSQRANALLANGMDLKDNQLVVPADGLYLIYSQVLFKGQGCPDYVLLTHTVSRFAISYQEKVSLLSAIKSPCPKDTPEGAELEPWYEPMYLGGVFQLEKGDLLSAEVNLPKYLDITESGQVYFGVIAL

SEQ ID NO: 23 - DNA, 23 bp, Artificial Sequence
  primer
tctcctggctgtgcctggagggc

SEQ ID NO: 24 - DNA, 24 bp, Artificial Sequence
  primer
ggcttgagcacagatcagcttcgg

SEQ ID NO: 25 - DNA, 23 bp, Artificial Sequence
  primer
tctcctggctgtgcctagagggc

SEQ ID NO: 26 - DNA, 24 bp, Artificial Sequence
  primer
ggcttgagcacggatgagcttcgg

SEQ ID NO: 27 - DNA, 22 bp, Artificial Sequence
  primer
ggaaaacattaagtcttgggtg

SEQ ID NO: 28 - DNA, 22 bp, Artificial Sequence
  primer
gtgaatgtccttgattaagggt

SEQ ID NO: 29 - DNA, 21 bp, Artificial Sequence
  primer
gtagttcatgctttcagccgt

SEQ ID NO: 30 - DNA, 20 bp, Artificial Sequence
  primer
agaagcccctctctgttgag

SEQ ID NO: 31 - DNA, 21 bp, Artificial Sequence
  primer
aggaggagaagttcccaaatg

SEQ ID NO: 32 - DNA, 21 bp, Artificial Sequence
  primer
ttgtcccttgaagagaacctg

SEQ ID NO: 33 - DNA, 18 bp, Artificial Sequence
  primer
ggtaggagacggcgatgc

SEQ ID NO: 34 - DNA, 20 bp, Artificial Sequence
  primer
caggcagtcagatcatcttc